SEQUENCE LISTING (60 sequences)

SEQ ID NO: 1 - DNA, 1251 bp, Oryza sativa
  source 1..1251: mol_type = genomic DNA
atggctgctgctgctgctgctgcgtcgtccgcgaagcgcgcgctcctcccgtgggcacgcgacgcccaccacgcgctggccagggccctgcagggatgcggcggcggcggcggcctcggtctccgcggggcgctcccgacggccggaggcaggtggtctctgctccagtgccggtggaggtcgtcgctgccgcagctcgactccgccgacaggtccgatgaggaaagcggcggcgaaatcgactgggacaacctggggttcgggctgacgccgaccgactacatgtacgtcatgcggtgctcgctggaggacggcgtcttctcccgcggcgagctcagccgctacggcaacatcgagctcagcccctcctccggcgtcatcaactacggccaggggctcttcgagggtctgaaggcgtacagggcggcgaaccaacaggggtcgtacatgctgttccggccggaggagaacgcgcggcggatgcagcacggcgccgagcgcatgtgcatgccgtcgccgtcggtggagcagttcgtccacgccgtcaagcagaccgtcctcgccaaccgccgctgggtgccaccgcaaggaaagggggcgctgtacatcaggccgctgctcatcgggagcggaccgattctcgggctggctcccgccccggagtacacgttcctcatctacgccgcaccggttggaacgtacttcaaggagggtctagcgccgataaaccttgtcgtagaggactcgatacaccgggccatgccgggcggcaccggcggggtcaagacgatcaccaactacgcgccggtgctcaaggcgcagatggacgccaagagcagagggttcactgacgtgctgtacctcgacgcggtgcacaagacgtacctggaggaggcctcctcctgcaacctcttcatcgtcaaggacggcgtcgtcgccacgccggccaccgtgggaaccatcctgccggggatcacgcgcaagagcgtcatcgagctcgccagggaccgcggctatcagcaggttgaagaacggctcgtctccatcgacgatctggtcggcgcagacgaggtgttctgcaccggaacagcggtggtcgttgccccagtatcgagtgttacttaccatgggcaaaggtacgagttcaggactggacatgacacgttatcgcagacactgcacacgactctgacgtccatccagatgggcctggctgaggacaagaaaggatggacagtggcaatagattaa

SEQ ID NO: 2 - protein, 416 aa, Oryza sativa
MAAAAAAASSAKRALLPWARDAHHALARALQGCGGGGGLGLRGALPTAGGRWSLLQCRWRSSLPQLDSADRSDEESGGEIDWDNLGFGLTPTDYMYVMRCSLEDGVFSRGELSRYGNIELSPSSGVINYGQGLFEGLKAYRAANQQGSYMLFRPEENARRMQHGAERMCMPSPSVEQFVHAVKQTVLANRRWVPPQGKGALYIRPLLIGSGPILGLAPAPEYTFLIYAAPVGTYFKEGLAPINLVVEDSIHRAMPGGTGGVKTITNYAPVLKAQMDAKSRGFTDVLYLDAVHKTYLEEASSCNLFIVKDGVVATPATVGTILPGITRKSVIELARDRGYQQVEERLVSIDDLVGADEVFCTGTAVVVAPVSSVTYHGQRYEFRTGHDTLSQTLHTTLTSIQMGLAEDKKGWTVAID

SEQ ID NO: 3 - DNA, 20 bp, synthetic construct
  source 1..20: mol_type = other DNA
cgggaggagcgcgcgcttcg

SEQ ID NO: 4 - DNA, 20 bp, synthetic construct
  source 1..20: mol_type = other DNA
cgcgctggccagggccctgc

SEQ ID NO: 5 - DNA, 35 bp, synthetic construct
  source 1..35: mol_type = other DNA
ttgctccgtggatccatggctgctgctgctgctgc

SEQ ID NO: 6 - DNA, 36 bp, synthetic construct
  source 1..36: mol_type = other DNA
aaagcggccgcaaatatctattgccactgtccatcc

SEQ ID NO: 7 - DNA, 20 bp, synthetic construct
  source 1..20: mol_type = other DNA
gtgcacaagacgtacctgga

SEQ ID NO: 8 - DNA, 20 bp, synthetic construct
  source 1..20: mol_type = other DNA
ggagacgagccgttcttcaa

SEQ ID NO: 9 - DNA, 20 bp, synthetic construct
  source 1..20: mol_type = other DNA
gcttccacaactccacacct

SEQ ID NO: 10 - DNA, 20 bp, synthetic construct
  source 1..20: mol_type = other DNA
cgcttgatcgttccacgaag

SEQ ID NO: 11 - DNA, 20 bp, synthetic construct
  source 1..20: mol_type = other DNA
gggaacactgcgactctacc

SEQ ID NO: 12 - DNA, 20 bp, synthetic construct
  source 1..20: mol_type = other DNA
gcccaacaggctggagtaat

SEQ ID NO: 13 - DNA, 20 bp, synthetic construct
  source 1..20: mol_type = other DNA
catgtggtggccatgggtat

SEQ ID NO: 14 - DNA, 20 bp, synthetic construct
  source 1..20: mol_type = other DNA
atctccagcaacctgctgaa

SEQ ID NO: 15 - DNA, 18 bp, synthetic construct
  source 1..18: mol_type = other DNA
catgccgtccaagatgca

SEQ ID NO: 16 - DNA, 20 bp, synthetic construct
  source 1..20: mol_type = other DNA
ggagtgcgacgacaggatgt

SEQ ID NO: 17 - DNA, 21 bp, synthetic construct
  source 1..21: mol_type = other DNA
ccgatatctatgcccattgga

SEQ ID NO: 18 - DNA, 20 bp, synthetic construct
  source 1..20: mol_type = other DNA
ccatccccctcttgatgaga

SEQ ID NO: 19 - DNA, 18 bp, synthetic construct
  source 1..18: mol_type = other DNA
gcagctcagcgagatgca

SEQ ID NO: 20 - DNA, 20 bp, synthetic construct
  source 1..20: mol_type = other DNA
cgttgatccgcatcgtgtac

SEQ ID NO: 21 - DNA, 20 bp, synthetic construct
  source 1..20: mol_type = other DNA
tcctacgccgacttctacca

SEQ ID NO: 22 - DNA, 20 bp, synthetic construct
  source 1..20: mol_type = other DNA
cggcgtaatccgcaaagaag

SEQ ID NO: 23 - DNA, 20 bp, synthetic construct
  source 1..20: mol_type = other DNA
caggttgagggagtcgtcac

SEQ ID NO: 24 - DNA, 20 bp, synthetic construct
  source 1..20: mol_type = other DNA
ggttgcctcagctacacctt

SEQ ID NO: 25 - DNA, 20 bp, synthetic construct
  source 1..20: mol_type = other DNA
gtgaaggctgttgttgtgct

SEQ ID NO: 26 - DNA, 20 bp, synthetic construct
  source 1..20: mol_type = other DNA
gccagagacacttccagtca

SEQ ID NO: 27 - DNA, 20 bp, synthetic construct
  source 1..20: mol_type = other DNA
tacttcccatcccgctacga

SEQ ID NO: 28 - DNA, 20 bp, synthetic construct
  source 1..20: mol_type = other DNA
tccttacatgctcggcttcg

SEQ ID NO: 29 - DNA, 20 bp, synthetic construct
  source 1..20: mol_type = other DNA
gccaagatccaggacaagga

SEQ ID NO: 30 - DNA, 18 bp, synthetic construct
  source 1..18: mol_type = other DNA
gccatcctccagctgctt

SEQ ID NO: 31 - DNA, 1155 bp, Glycine max
  source 1..1155: mol_type = genomic DNA
atgattcaaagaattgtgtactttcccagtttaaggaaattgcttcttcgggctggttctaattctgcttcgtccaagatcggaacgaacaattgctttgcttctcagccctcccctcttcggagtcacagcagtgatgacgagtatgctgatgtggactgggacagtcttggatttggactgatgaccactgattatatgtacattactaaatgttgtgagggccaaaattttggacaaggacaactcagtcgttatgggaacattgaactcagtccatcagctggggtcctaaattatggtcagggattattcgaaggcacgaaagcatacagaaaagaaaatgggggcttgctactcttccgtccagaagaaaatgccattcgcatgaagattggagccgaaagaatgtgcatggcatccccttccattgataattttgttgatgctttgaagcaaactgtattggctaataagcgttgggttcctccaccgggcaaaggatccttgtaccttaggcctctgctcctaggaactggtccagttttgggtttggctcctgcacctcaatacacattcctcatatttgcttcccctgttcgtaactatttcaaggagggctctgctccactcaacttgtacgtggaggagaactttgaccgtgcttctagccgcggcactggaaacgttaaaaccatttccaattatgcaccggtcttgatggcacaaattcaagccaagaaaagaggattttcggatgtgctataccttgattcagccacaaagaaaaatctggaggaggtctcttcttgtaacatttttattgccaagggcaaatgcatctcaacacctgctaccaatggaactattctttccggaattactcgaaaaagtgtcattgaaattgctcgtgatcatggctatcaggtagaagagcgtgttgttaacgtggatgaattaattgaggctgatgaagttttctgcacaggaactgcggtcggtgttgctccagtagggagtatcacataccaggataaaaggatggaatatataacaggttctggaaccatttgtcaagagttgaacaatacgatttcaggaattcaaacgggtactattgaagataagaagggatggattgttgaagttgattaa

SEQ ID NO: 32 - protein, 384 aa, Glycine max
MIQRIVYFPSLRKLLLRAGSNSASSKIGTNNCFASQPSPLRSHSSDDEYADVDWDSLGFGLMTTDYMYITKCCEGQNFGQGQLSRYGNIELSPSAGVLNYGQGLFEGTKAYRKENGGLLLFRPEENAIRMKIGAERMCMASPSIDNFVDALKQTVLANKRWVPPPGKGSLYLRPLLLGTGPVLGLAPAPQYTFLIFASPVRNYFKEGSAPLNLYVEENFDRASSRGTGNVKTISNYAPVLMAQIQAKKRGFSDVLYLDSATKKNLEEVSSCNIFIAKGKCISTPATNGTILSGITRKSVIEIARDHGYQVEERVVNVDELIEADEVFCTGTAVGVAPVGSITYQDKRMEYITGSGTICQELNNTISGIQTGTIEDKKGWIVEVD

SEQ ID NO: 33 - DNA, 1389 bp, Glycine max
  source 1..1389: mol_type = genomic DNA
atgcaatgttccaaaaaggagcaaaacactcgaggttgtataaatggctattcagattgtagagctggcgttgcccgttactcctcctatctatcgtgcgtatctctttctctctccatttttatttatatatttctctccattatttggtgtgcatgctctagacacacacacaccccaccaccaccaacactgagttttagcagaaggatagagagcagaatgattcaaagaactgtgtcctttcccagtttaaggaaattgcttcttcgggctggttgttctaaatctgcttcgtccaagatcggaacttacaattgctttgcttctcagtcctcccctctaccgagccacaaccctagttaccgtgatgacgagtatgctgatgtggactgggacagtcttggatttggactgatgcccactgattatatgtatattactaaatgttgtgagggccaaaattttggacaaggacaactcagtcgttatgggaacattgaactcagtccatcagctggtgtcctaaattatggtcagggtttattcgaaggcacgaaagcatacagaaaagaaaatgggggcttgctactcttccgtccagaagaaaatgccattcgcatgaagactggtgcccaaagaatgtgcatggcatcgccttccattgatcattttgttgatgctttgaagcaaactgtcttggctaataagcgttgggttcctccaccgggcaaaggatccttgtaccttaggcctctgctcctaggaactggtccggttttgggtttggctcctgcacctgaatacacattcctcatatttgcttcccctgttcgcaactatttcaaggagggctctgctccactcaacttgtacgtggaggaaaactttgaccgtgcttctagccgcggcactggaaacgttaaaaccatttccaattatgcaccggtcttgatggcacaaattcaagccaagaaaagaggattttcggatgtgctataccttgattcagacaccaagaaaaatctcgaggaggtctcttcttgtaacatttttattgccaagggcaaatgcatctcaacacctgctactaatggaactattctttccggaattacccgaaaaagtgtcattgaaattgctcgcgatcatggctatcaggtagaagagcgtgctgttgccgtggatgaattgattgaggctgatgaagttttctgcacaggaactgcggtcggtgttgctccagtagggagtatcacataccaggataaaaggatggaatatataacaggttctggaaccatttgtcaagagctgaacaataccatttcaggaattcaaacgggtactattgaagataagaagggatggattgtcgaagttgattaa

SEQ ID NO: 34 - protein, 462 aa, Glycine max
MQCSKKEQNTRGCINGYSDCRAGVARYSSYLSCVSLSLSIFIYIFLSIIWCACSRHTHTPPPPTLSFSRRIESRMIQRTVSFPSLRKLLLRAGCSKSASSKIGTYNCFASQSSPLPSHNPSYRDDEYADVDWDSLGFGLMPTDYMYITKCCEGQNFGQGQLSRYGNIELSPSAGVLNYGQGLFEGTKAYRKENGGLLLFRPEENAIRMKTGAQRMCMASPSIDHFVDALKQTVLANKRWVPPPGKGSLYLRPLLLGTGPVLGLAPAPEYTFLIFASPVRNYFKEGSAPLNLYVEENFDRASSRGTGNVKTISNYAPVLMAQIQAKKRGFSDVLYLDSDTKKNLEEVSSCNIFIAKGKCISTPATNGTILSGITRKSVIEIARDHGYQVEERAVAVDELIEADEVFCTGTAVGVAPVGSITYQDKRMEYITGSGTICQELNNTISGIQTGTIEDKKGWIVEVD

SEQ ID NO: 35 - DNA, 20 bp, Glycine max
  source 1..20: mol_type = genomic DNA
tttgaccgtgcttctagccg

SEQ ID NO: 36 - DNA, 21 bp, synthetic construct
  source 1..21: mol_type = other DNA
cgcgtttcgtttgtgcaaggt

SEQ ID NO: 37 - DNA, 20 bp, synthetic construct
  source 1..20: mol_type = other DNA
cagtatggcgtggtctccca

SEQ ID NO: 38 - DNA, 22 bp, synthetic construct
  source 1..22: mol_type = other DNA
aagaactgtgtcctttcccagt

SEQ ID NO: 39 - DNA, 20 bp, synthetic construct
  source 1..20: mol_type = other DNA
tagggttgtggctcggtaga

SEQ ID NO: 40 - DNA, 21 bp, synthetic construct
  source 1..21: mol_type = other DNA
gcttcttcgggctggttctaa

SEQ ID NO: 41 - DNA, 23 bp, synthetic construct
  source 1..23: mol_type = other DNA
atactcgtcatcactgtgactcc

SEQ ID NO: 42 - DNA, 20 bp, synthetic construct
  source 1..20: mol_type = other DNA
tgcagcgttttgggaacatc

SEQ ID NO: 43 - DNA, 20 bp, synthetic construct
  source 1..20: mol_type = other DNA
actcccatcttgtttgcggt

SEQ ID NO: 44 - DNA, 17 bp, synthetic construct
  source 1..17: mol_type = other DNA
agcgccgtctacggaag

SEQ ID NO: 45 - DNA, 21 bp, synthetic construct
  source 1..21: mol_type = other DNA
atgagaggaaactgcttgcga

SEQ ID NO: 46 - DNA, 20 bp, synthetic construct
  source 1..20: mol_type = other DNA
gttaggcaaccgcaaagacg

SEQ ID NO: 47 - DNA, 20 bp, synthetic construct
  source 1..20: mol_type = other DNA
cctcgtttcatgcgttgagc

SEQ ID NO: 48 - DNA, 21 bp, synthetic construct
  source 1..21: mol_type = other DNA
ggaacgtgctgtatcagtgga

SEQ ID NO: 49 - DNA, 22 bp, synthetic construct
  source 1..22: mol_type = other DNA
gtgttgcgcgtaattttgctga

SEQ ID NO: 50 - DNA, 20 bp, synthetic construct
  source 1..20: mol_type = other DNA
ggtttggcagagctggtttg

SEQ ID NO: 51 - DNA, 20 bp, synthetic construct
  source 1..20: mol_type = other DNA
ctttggacctgcttcccact

SEQ ID NO: 52 - DNA, 23 bp, Oryza sativa
  source 1..23: mol_type = genomic DNA
ccgcgttgcgcgcgctcctcccg

SEQ ID NO: 53 - RNA, 22 nt, synthetic construct
  source 1..22: mol_type = other RNA
ccgcgagcgcgcgctcctcccg

SEQ ID NO: 54 - RNA, 21 nt, synthetic construct
  source 1..21: mol_type = other RNA
ccgcgaagcgcgctcctcccg

SEQ ID NO: 55 - RNA, 22 nt, synthetic construct
  source 1..22: mol_type = other RNA
cgcgctggccagggcctgcagg

SEQ ID NO: 56 - RNA, 21 nt, synthetic construct
  source 1..21: mol_type = other RNA
tttgaccgtgcttctccgcgg

SEQ ID NO: 57 - RNA, 22 nt, synthetic construct
  source 1..22: mol_type = other RNA
tttgaccgtgcttctaccgcgg

SEQ ID NO: 58 - RNA, 20 nt, synthetic construct
  source 1..20: mol_type = other RNA
tttgaccgtgcttcccgcgg

SEQ ID NO: 59 - RNA, 11 nt, synthetic construct
  source 1..11: mol_type = other RNA
tttgaccgtgc

SEQ ID NO: 60 - RNA, 19 nt, synthetic construct
  source 1..19: mol_type = other RNA
tttgaccgtgcttccgcgg

(19) United States
(12) Patent Application Publication
(10) Pub. No.: US 2025/0270580 A1
(43) Pub. Date: Aug. 28, 2025

(54) BCAT gene controlling drought tolerance of plant and uses thereof

(71) Applicant: Lasemilla Co., Ltd., Gangwon-do (KR)

(72) Inventors: JU-KON KIM, Gangwon-do (KR); JUN SUNG SEO, Gangwon-do (KR); SE-JUN OH, Seoul (KR); HOBIN YOON, Gyeonggi-do (KR)

(21) Appl. No.: 19/179,557

(22) Filed: Apr. 15, 2025

Related U.S. Application Data
(63) Continuation-in-part of application No. 17/966,278, filed on Oct. 14, 2022, now abandoned.

(51) Int. Cl. C12N 15/82; A01H 1/06; C07K 14/415; C12N 9/22; C12N 15/11

(52) U.S. Cl. CPC C12N 15/8273; A01H 1/06; C07K 14/415; C12N 9/222; C12N 15/111; C12N 15/8201; C12N 2310/20

(57) ABSTRACT

A method of enhancing tolerance to drought stress in a plant includes inhibiting expression of a gene encoding a protein having the amino acid sequence of SEQ ID NO: 2 or genes encoding BCAT1 protein and BCAT2 protein from soybean having the amino acid sequence of SEQ ID NO: 32 and SEQ ID NO: 34, respectively in a plant cell.

### CROSS REFERENCE TO RELATED APPLICATIONS AND CLAIM OF PRIORITY

This application is a Continuation-In-Part of co-pending U.S. application Ser. No. 17/966,278, filed on Oct. 14, 2022 the entire disclosure of which is incorporated herein by reference for all purposes.

### GOVERNMENT LICENSE RIGHTS

This invention was made with Korea government support under the Cooperative Research Program for Agriculture Science & Technology Development (Project No. RS-2024-00398336), awarded by the Rural Development Administration, Republic of Korea.

### REFERENCE TO AN ELECTRONIC SEQUENCE LISTING

A sequence listing electronically submitted on Apr. 15, 2025 as a XML file named 20250415_Q98022GR07-CIP_TU_SEQ.XML, created on Apr. 15, 2025 and having a size of 58,620 bytes, is incorporated herein by reference in its entirety.

### BACKGROUND

### 1. Technical Field

The present invention relates to BCAT (BRANCHED-CHAIN AMINO ACID AMINOTRANSFERASE) gene controlling drought stress of plant and uses thereof.

This work was supported by the Starting growth Technological R&D Program (TIPS Program (No. S3307687)) funded by the Ministry of SMEs and Startups (MSS, Korea) in 2022.

### 2. Background Art

Unpredictable weather events caused by climate change occur worldwide. Abiotic stress is a natural incident with disastrous consequences for nearby living things. Moreover, extreme weather disasters lead to crop yield decreases. The drought stress significantly influenced international crop production. According to the reports, in rice (Oryza sativa. L.) plants, drought stress inflicts irreversible damage during the whole period of growth and decreases the grain yield drastically. Specifically, drought stress in the vegetative stage of rice (Yangliangyou 6, Hanyou 113) caused a yield reduction of 23-24%.

Plants have various strategies to combat abiotic stress. At the molecular level, plants regulate the expression of genes involving in stress responses through the stress-specific signaling pathway. Physiological responses lead to morphological and endogenous biochemical adjustments. For instance, plant amino acids play an essential role in minimizing the damage by abiotic stress. So far, proline is best characterized amino acid that accumulates in plant cells to confront a wide range of abiotic stress. It has been documented that proline accumulates in response to drought, salt, heat, low temperature, and nutrient deficient stress in various plants. γ-Aminobutyric acid (GABA) alleviates drought and heat stress through regulating antioxidant genes in sunflower. Besides, glycine, taurine, arginine, and serine were reported as stress-induced amino acids which act as osmolytes. Interestingly, among plant amino acids, Branched-chain amino acids (BCAA), leucine (Leu), isoleucine (Ile), and valine (Val) have been reported to accumulate under stress conditions in plants. Fold change value of BCAA quantity under drought conditions was higher than that of proline, a well-known drought inducible amino acid.

BCAA has a critical role in protein biosynthesis and plant development.

Moreover, they are also involved in the formation of diverse secondary metabolites, such as cyanogenic glycosides, glucosinolate, and acyl-sugars. Branched-chain amino acids synthesis is carried out with two distinct substrates. The preliminary pathway of valine and leucine starts with pyruvate created by glycolysis. On the other hand, the synthesis of isoleucine starts with 2-ketobutyrate after the threonine deamination by threonine deaminase (TD, EC 4.2.1.16). The first engaged enzyme for Val, Leu production, is acetohydroxyacid synthase (AHAS also known as acetolactate synthase ALS, EC 2.2.1.6), which catalyzes both two pyruvate molecules for the constitution of acetolactate for Val and α-ketobutyrate with one pyruvate to form acetohydroxybutyrate as a precursor of isoleucine. Ketol-acid reductoisomerase (KARI, EC 1.1.1.86), and dihydroxy-acid dehydratase (DHAD, EC 4.2.1.9), consecutively catalyze a fundamental two-step reaction for BCAA synthesis. The branched-chain amino acid aminotransferase (BCATs, EC 2.6.1.42) is in charge of the final step of BCAA biosynthesis in plastids.

BCAA catabolic reactions mainly occur in mitochondria. BCAT is an enzyme involved in the first step of degradation and the last step in synthesizing BCAA. BCAT, which belongs to Class IV of the pyridoxal 5′phosphate-dependent aminotransferases, converts Val, Ile, or Leu to α-keto acids KIV, KIC, or KMV. Branched-chain keto acid dehydrogenase (BCKDH also known as BCKDH complex, EC 1.2.4.4) catalyzes the branched-chain α-keto acids to isobutyryl-CoA, isovaleryl-CoA, or methylbutyryl-CoA (Taylor et al. 2004). Then, Isovaleryl-CoA dehydrogenase (IVD, EC 1.3.99.10), identified in several plant species, catalyzes acyl-CoA to enoyl-CoA. Although this enzyme has not been fully elucidated, the substrate specificity appears to be different among these proteins of other species.

Among these catabolic enzymes, Interestingly, plant species have various BCAT isoform families. In Arabidopsis thaliana and Solanum lycopersicum, seven and six distinct isoforms of BCAT have been validated, respectively. It has been reported that AtBCAT2 (At1g10070), S1BCAT1 (SGN-U569828), and S1BCAT2 (SGN-U569830) are localized to mitochondria, where it conducts a vital function in BCAA catabolism Nevertheless, the pathways related to gene and catabolic protein networks have not been fully elucidated in plants.

Meanwhile, in Korean Patent Publication No. 2021-0063574, “Drought resistant transgenic plant inhibiting AtPR5K2 protein expression” is disclosed, and in Korean Patent Publication No. 2022-0083464, “Novel branched-chain amino acid aminotransferase variant and method of producing L-isoleucine using the same” is disclosed. However, BCAT gene controlling plant drought tolerance of the present invention and uses thereof have not been described.

### SUMMARY

The present invention is devised according to the necessities that are explained in the above. Specifically, based on previous research examining the role played by branched-chain amino acids (BCAA) on plant tolerance under abiotic stress conditions, inventors of the present invention studied the function of OsBCAT2 (BRANCHED-CHAIN AMINO ACID AMINOTRANSFERASE 2) gene, which is derived from rice.

By using CRISPR-Cas9 system, the inventors of the present invention prepared loss of function rice OsBCAT2 mutants (i.e., osbcat2). In addition, as a result of examining the drought tolerance exhibited by the osbact2. the inventors found that osbact2 mutants show enhanced tolerance to drought and enhanced grain yield over those of wild type under various drought stress conditions. Also, after finding out soybean-derived BCATs with more than 50% amino acid homology to rice-derived BCAT2 in the soybean genome, dual loss-of-function mutants (Gmbcat1/2) of soybean BCAT1 and BCAT2 were manufactured using the CRISPR-Cas9 system to confirm their drought stress resistance, and it was confirmed that the Gmbcat1/2 mutant showed improved drought resistance and agricultural yield compared to the wild type. The present invention is completed accordingly.

To solve the problems that are described in the above, the present invention provides a method of enhancing tolerance to drought stress in a plant comprising inhibiting expression of a gene encoding BCAT (BRANCHED-CHAIN AMINO ACID AMINOTRANSFERASE) protein derived from plant in a plant cell.

The present invention further provides a method of producing a transgenic plant having controlled tolerance to drought stress comprising: transforming a plant cell with a recombinant vector containing a gene encoding BCAT protein derived from plant: and regenerating a transgenic plant from the transformed plant cell.

The present invention further provides a transgenic plant having controlled tolerance to drought stress produced by the aforementioned method of producing a transgenic plant, and a transgenic seed thereof.

The present invention further provides a method of producing a genome-edited rice plant having enhanced tolerance to drought stress comprising: (a) carrying out genome editing by introducing guide RNA specific to target nucleotide sequence in a gene encoding OsBCAT2 protein derived from rice, and endonuclease protein to a rice plant cell: (b) producing a rice plant cell comprising an edited nucleotide sequence; and (c) regenerating a plant from the genome-edited rice plant cell.

The present invention further provides a genome-edited rice plant having enhanced tolerance to drought stress produced by the aforementioned method of producing a genome-edited rice plant, and a genome-edited seed thereof.

The present invention further provides a method of producing a genome-edited soybean plant having enhanced tolerance to drought stress comprising: (a) carrying out genome editing by introducing guide RNA specific to target nucleotide sequence in genes encoding BCAT1 protein and BCAT2 protein from soybean and an endonuclease protein to a soybean plant cell: (b) producing a soybean plant cell comprising an edited nucleotide sequence: and (c) regenerating a plant from the genome-edited soybean plant cell.

The present invention further provides a genome-edited soybean plant having enhanced tolerance to drought stress produced by the aforementioned method of producing a genome-edited soy bean plant, and a genome-edited seed thereof.

Because having crops with tolerance to environmental stress is considered to be very important in the field of agriculture, it is expected that OsBCAT2 (branched-chain amino acid aminotransferase 2) gene derived from rice and BCAT1 and BCAT2 genes from soybean, which are described in the present invention, can be advantageously used for enhancing the plant tolerance to drought stress.

### BRIEF DESCRIPTION OF THE DRAWINGS

FIGS. 1A and 1B show expression patterns of OsBCAT2 in response to various abiotic stress and hormones. FIG. 1A The relative expression level of OsBCAT2 under drought (air-drying), Salt (400 mM NaCl), low temperature (4° C.). FIG. 1B The relative expression level of OsBCAT2 after treatment with 100 μM of abscisic acid, methyl jasmonate, and salicylic acid, respectively. The rice uibiquitin1 (Os06g068100) was used as an internal standard for normalization. The error bar indicates the standard deviation of three replicate measurements.

FIGS. 2A and 2B show osmotic stress phenotype induced by PEG after FAA (free amino acid) treatment.

FIGS. 3A to 3C show the phenotype of FAA-treated NT plants induced by air drying. FIG. 3A Air-drying induced abiotic stress phenotype (n=10). FIG. 3B Relative water content change by time-course during air-dry treatment after amino acid treatment in the NT plant. FIG. 3C Total water loss during air-drying.

FIG. 4 shows the expression pattern of OsBCAT2 genes at various developmental stages and tissue. Total RNA was extracted at various tissue and developmental stages (d, day: w, week: m, month: M, meiosis; BH, before heading: AH, after heading). The expression level was analyzed by quantitative RT-PCR analysis of OsBCAT2. The rice UIBIQUITIN1 (Os06g068100) was used as an internal standard for normalization. The error bar indicates the standard deviation of three replicate measurements.

FIGS. 5A and 5B show subcellular localization of the OsBCAT2 protein in rice protoplasts. 35S::OsBCAT2-GFP and CD3-991-mCherry as mitochondria markers were co-transfected and transiently expressed in rice leaf protoplasts. Scale bar, 10 μm

FIGS. 6A and 6B show construction for loss of function OsBCAT2 by CRISPR-Cas9 systems and their genotype. FIG. 6A Two single guide RNA were designed in the first exon region to derive the premature stop codon. FIG. 6B Mutation pattern on osbcat2 mutant #3, #4, #5, #10 and #15. Underlined marking stands for protospacer adjacent motif (PAM), and base deletion is marked with a hyphen.

FIGS. 7A to 7E show loss of function OsBCAT2 confers drought tolerance. FIG. 7A Five different osbcat2 lines were used. Visual results from sequential drought stress and re-watered treatments in the greenhouse under normal conditions for a month, including non-transgenic (NT) plants. The number of days on the images indicates the duration of the drought and re-watering. FIG. 7B Photochemical efficiency measured during the drought treatment period, Fv/Fm values indicates the quantum yield of photosystem II. FIG. 7C Performance index shows the efficiency of the two photosystems (I and II) carrying out potential photosynthesis of plant leaves (n=10). FIG. 7D Survival rates were counted after recovery via re-watering after drought treatment. FIG. 7E Soil moisture during the time-course drought treatment period (n=20). Asterisks denote statistically significant differences compared to NT unpaired student's t-tests as follows: *P<0.05

FIGS. 8A and 8B show loss of OsBCAT2 accumulated BCAA content under various drought conditions in shoot and roots. FIG. 8A BCAA content in osbcat2 and NT before and after air dry treatment. FIG. 8B BCAA content accumulation by time-course drought stress in osbcat2 and NT. The error bar indicates the standard deviation, the technical repeat of each experiment was repeated three times. Asterisks denote statistically significant differences compared to NT unpaired student's t-tests as follows: *P<0.05, **P<0.01, ****P<0.0001.

FIGS. 9A and 9B show the yield of osbcat2 improved under drought conditions. The agronomic trait of osbcat2 and NT in FIG. 9A normal and FIG. 9B drought field conditions. Mean values from NT plants in Table 3 were allocated a reference value of 100%. CL, culm length: PL, panicle length: NP, number of panicles; NS, number of spikelets; NTS, number of total spikelets; FR, filling rate: TGW, total grain weight: 1000 GW, 1000-grain weight.

FIGS. 10A to 10C show transcription analysis of JA-related gene in drought time course treatment in greenhouse conditions of osbcat2 and NT plants. Relative expression of FIG. 10A a critical enzyme for JA-Ile formation, FIG. 10B JA-response gene and FIG. 10C JA-signaling core genes. The rice ubiquitin1 (Os06g068100) was used as an internal standard for normalization. The error bar indicates the standard deviation of three replicate measurements. Asterisks denote statistically significant differences compared to NT unpaired student's t-tests as follows: *P<0.05, **P<0.01, ****P<0.0001

FIGS. 11A to 11D show loss of function OsBCAT2 enhances the reactive oxygen species (ROS)-scavenging capacity under time-course water deficit conditions. FIG. 11A The histochemical staining assay through 3,3-diaminobenzidine (DAB) and nitro blue tetrazolium (NBT) for the detection of H₂O₂ and O₂ in osbcat2 compared to NT under drought conditions, and FIG. 11B, FIG. 11C its stained area. FIG. 11D Relative expression of antioxidant genes, L-ASCORBATE PEROXIDASE2 (APX2), CATALASE (CAT), and SUPEROXIDE DISMUTASE1.2 (SOD1.2). The rice UIBIQUITIN 1 (Os06g068100) was used as an internal standard for normalization. The error bar indicates the standard deviation of three replicate measurements. Asterisks denote statistically significant differences compared to NT unpaired student's t-tests as follows: *P<0.05, **P<0.01, ****P<0.0001

FIG. 12 shows that seven soybean BCAT-encoding genes (GmBCAT1, 2, 3, 4, 5, 6, and 7) with amino acid sequence homology of 50% or more with rice-derived BCAT2 were discovered in the soybean genome. The amino acid sequence similarity was analyzed through the phylogenetic tree analysis results between soybean BCAT gene and Arabidopsis thaliana and tomato BCAT genes.

FIG. 13 shows the results of determining the expression changes of the seven BCAT genes in leaves isolated from soybean after drought treatment. It was found that the expression of BCAT1 and BCAT2 is enhanced 5 to 7 days after the drought treatment.

FIG. 14 shows the results of determining the expression changes of the seven BCAT genes in leaves/stems/root tissues isolated from soybean after salt (150 mM NaCl) treatment. It was found that the expression of BCAT1 and BCAT2 is significantly enhanced in leaf tissues 24 hours after the salt treatment.

FIG. 15 shows the results of determining the expression changes of the seven BCAT genes in leaves/stems/root tissues isolated from soybean after ABA (100 μM) treatment. It was found that the expression of BCAT1 and BCAT2 is significantly enhanced in root tissues 6 hours after the ABA treatment.

FIGS. 16A and 16B show the subcellular localization of the seven soy bean-derived BCAT genes in rice protoplast, which has been co-transformed with the 35S::GmBCAT-YFP vector and CD3-991-mCherry as a mitochondrial marker and transiently induced to express them. Size bar, 10 μm.

FIGS. 17A and 17B show the loss-of-function constructs and genotypes of GmBCAT1 and GmBCAT2, in which the loss-of-function was achieved by using CRISPR-Cas9 system. FIG. 17A shows that a single guide RNA was designed to induce a premature stop codon in the fifth exon region of GmBCAT1, and FIG. 17B shows that a single guide RNA was designed to induce a premature stop codon in the sixth exon region of GmBCAT2. The underlined indicates PAM, and the base deletion is indicated by a hyphen.

FIGS. 18A to 18C show that loss of function of GmBCAT1 and GmBCAT2 confers drought tolerance. In FIG. 18A, two different Gmbcat1/2 lines were used, and the figure shows plants which had been sequentially subjected to drought stress and re-watering treatment for one month in a greenhouse under normal conditions, including NT plants. The number of days indicated in the photographic images indicates the drought treatment and re-watering periods. FIG. 18B shows soil moisture over time during the drought treatment period (n=20). FIG. 18C is the calculated survival rate after recovery by re-watering after the drought treatment. The asterisk in FIG. 18C indicates significance compared to NT, as analyzed by unpaired student's t-tests, **p<0.01.

FIGS. 19A and 19B show that the yield of Gmbcat1/2 mutant was enhanced under drought stress conditions. Specifically, agronomic characteristics of NT plants and Gmbcat1/2 mutants, either FIG. 19A under normal conditions or FIG. 19B under drought conditions, are shown. The asterisk in FIG. 19B indicates significance compared to NT as analyzed by unpaired student's t-tests, **p<0.01.

FIG. 20 shows the contents of valine (Val), isoleucine (Ile), and leucine (Leu) as BCAA and the content of proline (Pro) in leaves of NT plants and Gmbcat1/2 mutants. Error bars represent standard deviations based on three replicates, and asterisks represent significance compared to NT as analyzed by unpaired student's t-tests, *p<0.05.

### DETAILED DESCRIPTION

To achieve the object of the present invention, the present invention provides a method of enhancing tolerance to drought stress in a plant comprising inhibiting expression of a gene encoding BCAT (branched-chain amino acid aminotransferase) protein derived from plant in a plant cell.

With regard to the method of enhancing tolerance to drought stress according to one embodiment of the present invention, the BCAT protein from plant may be BCAT2 protein from rice, or BCAT1 protein and BCAT2 protein from soybean, but it is not limited thereto.

The OsBCAT2 protein derived from rice may preferably consist of the amino acid sequence of SEQ ID NO: 2 and the BCAT1 protein and BCAT2 protein from soy bean may preferably consist of the amino acid sequence of SEQ ID NO: 32 and SEQ ID NO: 34, respectively, but it is not limited thereto.

Also included in the scope of the OsBCAT2 protein derived from rice and BCAT1 protein and BCAT2 protein from soybean according to the present invention are the protein having the amino acid sequence represented by SEQ ID NO: 2, SEQ ID NO: 32 and SEQ ID NO: 34, respectively and functional equivalents thereof. As described herein, the term “functional equivalents” means a protein which has, as a result of addition, substitution, or deletion of an amino acid, at least 70%, preferably at least 80%, more preferably at least 90%, and even more preferably at least 95% sequence homology with the amino acid sequence represented by SEQ ID NO: 2, SEQ ID NO: 32 and SEQ ID NO: 34, respectively and it indicates a protein which exhibits substantially the same physiological activity as the protein represented by SEQ ID NO: 2, SEQ ID NO: 32 and SEQ ID NO: 34, respectively. The expression “substantially the same physiological activity” indicates an activity of controlling plant tolerance to drought stress.

Also included in the present invention is a gene which encodes OsBCAT2 protein derived from rice and BCAT1 protein and BCAT2 protein from soybean, and the gene has the scope to encompass all genomic DNA, cDNA, and synthetic DNA which encode OsBCAT2 protein derived from rice and BCAT1 protein and BCAT2 protein from soy bean. Preferably, the gene encoding OsBCAT2 protein derived from rice described in the present invention may comprise the OsBCAT2 nucleotide sequence derived from rice that is represented by SEQ ID NO: 1. Also, the gene encoding BCAT1 protein and BCAT2 protein derived from soybean described in the present invention may comprise the BCAT1 and BCAT2 nucleotide sequences derived from soy bean that is represented by SEQ ID NO: 31 and SEQ ID NO: 33, respectively.

Furthermore, homologs of the aforementioned sequence are also within the scope of the present invention. Specifically, the aforementioned gene may include a nucleotide sequence which has 70% or higher, more preferably 80% or higher, even more preferably 90% or higher, and most preferably 95% or higher sequence homology with the nucleotide sequence of SEQ ID NO: 1, SEQ ID NO: 31 or SEQ ID NO: 33. The “sequence homology %” of the polynucleotide is identified by comparing two sequences that are optimally aligned. In this regard, a part of the polynucleotide in comparative region may comprise an addition or a deletion (i.e., a gap) compared to a reference sequence (without any addition or deletion) in the optimized alignment of the two sequences.

The method of enhancing plant tolerance to drought stress according to the present invention may involve the inhibition of expression of a gene encoding BCAT protein derived from plant in plant cell so as to have higher plant tolerance to drought stress compared to a non-transgenic plant, but it is not limited thereto.

According to one embodiment of the present invention, the inhibition of expression of a gene encoding BCAT protein derived from plant can be an inhibition (suppression) of the gene encoding BCAT protein derived from plant based on insertion of T-DNA to the gene encoding BCAT protein derived from plant, induction of mutation using endogenous transposon or irradiation of X-ray or y-ray, or use of RNAi or antisense RNA, or CRISPR/Cas9 gene editing system, but it is not limited thereto. Any method typically employed in the pertinent art to have inhibited gene expression can be possibly used.

With regard to the recombinant vector of the present invention, the promoter is a promoter which is suitable for transformation. Preferably, it may be any one of CaMV 35S promoter, actin promoter, ubiquitin promoter, pEMU promoter, MAS promoter or histone promoter. Preferably, it may be CaMV 35S promoter, but it is not limited thereto.

The recombinant expression vector may preferably contain one or more selective marker. The selective marker is a nucleotide sequence having a property of allowing vector selection by a common chemical method. Any gene that can be used for identifying transformed cells from non-transformed cells can be a selective marker. The marker gene can be a dominant drug-resistance gene, but it is not limited thereto.

In case of transforming an eukaryotic cell with the vector of the present invention, yeast (Saccharomycescerevisiae), an insect cell, human cell (e.g., CHO (Chinese hamster ovary) cell line, W138, BHK, COS-7, 293, HepG2, 3T3, RIN and MDCK cell line), a plant cell, or the like can be used as a host cell. Preferably, the host cell is a plant cell.

The present invention further provides a method of producing a transgenic plant having controlled tolerance to drought stress comprising: transforming a plant cell with a recombinant vector containing a gene encoding BCAT protein derived from plant: and regenerating a transgenic plant from the transformed plant cell.

With regard to the method of producing a transgenic plant according to the present invention, the BCAT protein derived from plant may preferably include a BCAT2 protein from rice consisting of the amino acid sequence of SEQ ID NO: 2, a BCAT1 protein from soybean consisting of the amino acid sequence of SEQ ID NO: 32, or a BCAT2 protein from soybean consisting of the amino acid sequence of SEQ ID NO: 34 and functional equivalents thereof. In this regard, the detailed information is the same as those described above.

Further, with regard to the method of producing a transgenic plant described in the present invention, the method for transforming a plant cell is the same as described in the above. As for the method for regenerating a transgenic plant from a transformed plant cell, a method well known in the pertinent art can be used. The transformed plant cell needs to be regenerated into a mature plant. Techniques for regeneration into a mature plant by culture of callus or protoplast are well known in the pertinent art for various species.

With regard to the method of producing a transgenic plant according to one embodiment of the present invention, by inhibiting the expression of the gene, which encodes BCAT protein derived from plant, in plant cell, higher plant tolerance to drought stress compared to a non-transgenic plant, i.e., wild type, can be obtained, but it is not limited thereto.

Inhibiting the expression of the gene which encodes BCAT protein derived from plant can be achieved by transforming a plant cell with a recombinant vector containing sense or antisense DNA, or microRNA for BCAT gene derived from plant to inhibit the expression of the gene which encodes BCAT protein derived from plant. However, the inhibition is not limited thereto, and any gene expression inhibition technique well known in the pertinent art can be employed.

The present invention further provides a transgenic plant having controlled tolerance to drought stress, which is produced by the aforementioned method of producing a transgenic plant, and a transgenic seed thereof.

The transgenic plant according to the present invention is characterized in that, as expression of the gene encoding BCAT protein derived from plant is inhibited, the plant exhibits enhanced tolerance to drought stress. The plant can be a monocot plant such as rice, barley, wheat, rye, corn, sugar cane, oat, or onion, or a dicot plant such as Arabidopsis thaliana, potato, eggplant, tobacco, pepper, tomato, burdock, crown daisy, lettuce, balloon flower, spinach, chard, yam, carrot, water parsley, Chinese cabbage, cabbage, Raphanus sativus for. raphnistroides MAK, watermelon, oriental melon, cucumber, zucchini, gourd, strawberry, soybean, mung bean, kidney bean, or sweet pea. Preferably, it can be rice or soybean plant. However, it is not limited thereto.

The present invention further provides a method of producing a genome-edited rice plant having enhanced tolerance to drought stress comprising: (a) carrying out genome editing by introducing guide RNA specific to target nucleotide sequence in a gene encoding OsBCAT2 protein derived from rice, and enconuclease protein to a rice plant cell: (b) producing a rice plant cell comprising an edited nucleotide sequence: and (c) regenerating a plant from the genome-edited rice plant cell.

As described herein, the term “genome/gene editing” means a technique for introducing a target-oriented mutation to a genome nucleotide sequence of a plant or an animal cell including human cell. Specifically, it indicates a technique for knock-out or knock-in of a specific gene by deletion, insertion, or substitution of one more nucleic acid molecules by DNA cutting, or a technique for introducing a mutation even to a non-coding DNA sequence which does not produce any protein. According to the purpose of the present invention, the genome editing may be an introduction of a mutation to a plant by using an endonuclease, for example, Cas9 (CRISPR associated protein 9) protein, and a guide RNA. Furthermore, the term “gene editing” may be interchangeably used with “gene engineering”.

Furthermore, the term “target gene” described herein means part of DNA which is present in the genome of a plant and to be edited according to the present invention. Type of the target gene is not limited, and it may include a coding region and also a non-coding region. Depending on the purpose, a person who is skilled in the pertinent art may select the target gene according to the mutation of a plant that is desired to be produced by genome editing.

Furthermore, the term “guide RNA” described herein means a ribonucleic acid which includes RNA specific to a target DNA in nucleotide sequence encoding the target gene and, according to complementary binding between the whole or partial sequence of the guide RNA and the nucleotide sequence of target DNA, the guide RNA plays the role of guiding an endonuclease to the target DNA nucleotide sequence. The guide RNA represents two types of RNA, i.e., a dual RNA having crRNA (CRISPR RNA) and tracrRNA (trans-activating crRNA) as a constitutional element: or a single chain guide RNA (sgRNA) comprising the first site which includes a sequence that is fully or partially complementary to the nucleotide sequence in the target gene and the second site which includes a sequence interacting with RNA-guide nuclease, but those in the form in which the RNA-guide nuclease is active for the target nucleotide sequence are also within the scope of the invention without any limitation. Considering the type of an endonuclease to be used together, microorganisms from which the endonuclease is derived, or the like, the guide RNA may be suitably selected according to the technique that is well known in the pertinent art.

Furthermore, the guide RNA may be those transcribed from a plasmid template, those obtained by in vitro transcription (e.g., oligonucleotide double strand), or those obtained by synthesis, or the like, but it is not limited thereto.

Furthermore, with regard to the method of producing a genome-edited rice plant according to the present invention, the endonuclease protein may be one or more selected from a group consisting of Cas9, Cpf1 (CRISPR from Prevotella and Francisella 1), TALEN (Transcription activator-like effector nuclease), ZFN (Zinc Finger Nuclease), and a functional analog thereof. It may be preferably Cas9 protein, but it is not limited thereto. Furthermore, the Cas9 protein may be one or more selected from a group consisting of Cas9 protein derived from Streptococcus pyogenes, Cas9 protein derived from Campylobacter jejuni, Cas9 protein derived from S. thermophilus, Cas9 protein derived from S. aureus, Cas9 protein derived from Neisseria meningitides, Cas9 protein derived from Pasteurella multocida, Cas9 protein derived from Francisella novicida, and the like, but it is not limited thereto. Information of Cas9 protein or gene of Cas9 protein can be obtained from a known database like GenBank of NCBI (National Center for Biotechnology Information).

Cas9 protein is an RNA-guided DNA endonuclease enzyme which induces breakage of a double stranded DNA. In order for Cas9 protein to cause DNA breakage after precise binding to a target nucleotide sequence, a short nucleotide sequence consisting of three nucleotides, which is known as PAM (Protospacer Adjacent Motif), should be present next to the target nucleotide sequence, and Cas9 protein causes the breakage by assuming the position between the 3^{rd} and the 4^{th} base pairs from the PAM sequence (NGG).

In the present invention, the guide RNA and endonuclease protein may function as RNA gene scissors (RNA-Guided Engineered Nuclease, RGEN) by forming a ribonucleic acid-protein (i.e., ribonucleoprotein) complex.

The CRISPR/Cas9 system employed in the present invention is a method of gene editing based on NHEJ (non-homologous end joining) mechanism in which insertion-deletion (InDel) mutation resulting from incomplete repair, which is induced during a process of DNA repair, is caused by introducing breakage of a double strand at specific site of a specific gene to be edited.

With regard to the method of producing a genome-edited rice plant according to the present invention, introducing guide RNA and endonuclease protein to a rice plant cell of the aforementioned step (a) may involve use of: a complex (i.e., ribinucleoprotein) of a guide RNA specific to the target nucleotide sequence in OsBCAT2 gene derived from rice and an endonuclease protein: or a recombinant vector having a DNA encoding a guide RNA specific to the target nucleotide sequence in OsBCAT2 gene derived from rice and a nucleic acid sequence encoding an endonuclease protein, but it is not limited thereto.

Furthermore, with regard to the method of producing a genome-edited rice plant according to one embodiment of the present invention, the target nucleotide sequence in OsBCAT2 gene derived from rice may be the nucleotide sequence of SEQ ID NO: 3 or SEQ ID NO: 4, but it is not limited thereto.

The present invention further provides a genome-edited rice plant having enhanced tolerance to drought stress, which is produced by the aforementioned method of producing a genome-edited rice plant, and a genome-edited seed thereof.

The genome-edited rice plant according to the present invention is characterized in that, as the gene encoding BCAT2 protein derived from rice is knocked-out, the plant exhibits enhanced tolerance to drought stress.

The present invention further provides a method of producing a genome-edited soy bean plant having enhanced tolerance to drought stress comprising: (a) carrying out genome editing by introducing guide RNA specific to target nucleotide sequence in genes encoding BCAT1 (branched-chain amino acid aminotransferase 1) protein and BCAT2 protein from soybean and an endonuclease protein to a soybean plant cell: (b) producing a soy bean plant cell comprising an edited nucleotide sequence; and (c) regenerating a plant from the genome-edited soybean plant cell.

The present invention further provides a genome-edited soybean plant having enhanced tolerance to drought stress, which is produced by the aforementioned method of producing a genome-edited soybean plant, and a genome-edited seed thereof.

Furthermore, with regard to the method of producing a genome-edited soy bean plant according to one embodiment of the present invention, introducing guide RNA and an endonuclease protein to a soybean plant cell in step (a) is the same as the method for producing the above-described genome-edited rice plant.

Furthermore, with regard to the method of producing a genome-edited soybean plant according to one embodiment of the present invention, the target nucleotide sequence in BCAT1 and BCAT2 gene from soybean may be the nucleotide sequence of SEQ ID NO: 35, but it is not limited thereto.

Hereinbelow, the present invention is explained in greater detail in view of the Examples. However, it is evident that the following Examples are given only for exemplification of the present invention and by no means the present invention is limited to the following Examples.

### Materials And Method

### 1. Plant Materials

To generate a loss of function OsBCAT2 (Os03g0231600, LOC_Os03g12890) mutants (osbcat2), rice (Oryza sativa L. ssp. Japonica cv. Dongjin) was edited using the CRISPR-Cas9 system through two guide RNAs. Guide RNA was designed via the web-based tool CRISPR RGEN Tools. As described previous report, the method was performed (Chung et al. 2020, Int. J. Mol. Sci. 21 (24);9606). We obtained osbcat2 mutants with a total of 18 genotypes. Among these, we selected osbcat2 mutants (#3, #4, #5, #10, and #15), which have premature stop codons before 89 amino acids were used in this research.

The Japonica rice cultivar Dongjin (Oryza sativa L.) grown under the same conditions as mutants (osbcat2), was used as the non-transgenic control.

TABLE 1
sgRNAs for OsBCAT2 gene editing

| | Sequence (5′-3′) |
|---|---|
| sgRNA1 | CGGGAGGAGCGCGCGCTTCG (SEQ ID NO: 3) |
| sgRNA2 | CGCGCTGGCCAGGGCCCTGC (SEQ ID NO: 4) |

To generate loss-of-function mutants (Gmbat1/2) of GmBCAT1 (Glyma.04G049200) and GmBCAT2 (Glyma.06G050100), soybean (Glycine max var. Williams 82) was edited by the CRISPR-Cas9 system using a single guide RNA (Table 2). The guide RNA was designed using the web-based tool CRISPR RGEN Tools (http://www.rgenome.net). Soybean transformation was performed as described in a previous report, and a total of 41 transformants were obtained, and 15 transformants with edited gene were selected. Among them, Gmbat1 2 mutants (#1, #18) having a premature stop codon were used in the present invention.

Soybean Williams82 variety grown under the same conditions as the mutant (Gmbcat1/2) was used as a non-transformed control (NT).

TABLE 2
sgRNA for Gene editing of GmBCATI and GmBCAT2

| | Sequence (5′-3′) |
|---|---|
| sgRNA1 | TTTGACCGTGCTTCTAGCCG (SEQ ID NO: 35) |

### 2. Abiotic Stress and Phytohormone Treatments

1) Rice

On a Murashige-Skoog (MS) solid medium, rice seeds (Oryza sativa cv. Dongjin) were germinated for four days at 28° C. in the dark. The seedlings were then transported to a growth chamber with a 16 h light/8 h dark cycle, 200 μmol m⁻² s-1 of light intensity, and 70% relative humidity. One-week-old seedlings were transferred to a Yoshida solution and grown for two more weeks for gene expression analysis. Non-transgenic (NT) (Oryza sativa L. ssp. Japonica cv. Dongjin) seedlings were used to study the phytohormone-dependent response of OsBCAT2 and were transferred to 50 ml tubes containing 100 μM ABA (abscisic acid), JA (jasmonic acid), and SA (salicylic acid) solution (Sigma, USA). Seedlings were harvested every 2 hours until 6 hours after each hormone treatment for RNA extraction.

To confirm the transcription levels of the OsBCAT2 gene under various abiotic stresses, non-transgenic (NT) plants were grown in soil for two weeks under standard greenhouse conditions (16 h light/8 h dark cycles at 28˜30° C.). For stress treatments, remove the soil from the roots of the seedling completely, and drought stress was induced by air-drying the seedlings, while salinity stress was imposed by incubating the seedlings in water containing 400 mM NaCl at 28° C. Low-temperature stress was treated by incubating the seedlings in water at 4° C. (+1.5° C.).
2) Soy Bean

Soybean seeds (Glycine max var. Williams 82) were germinated directly in soil, and the seedlings were grown in a greenhouse at 25° C., a photoperiod of 16 h light/8 h dark, a light intensity of 200 μmol m⁻²s⁻¹, and a relative humidity of 50%. Non-transgenic (NT) seedlings were used to study the phytohormone response of GmBCATs, and 100 μM ABA was sprayed on the leaves. For RNA extraction, the seedlings were sampled at each time point up to 24 hours after each hormone treatment.

To determine the transcription levels of GmBCATs genes under various abiotic stresses, NT plants were grown in soil for 4 weeks under standard greenhouse conditions (16-h light/8-h dark cycle at 25° C.). Drought stress was applied by withholding water, and salinity stress was applied by supplying water containing 200 mM NaCl at 28° C.

### 3. Amino Acid Feeding and Water Deficit Stress Treatment

Non-transgenic plants (Oryza sativa cv. Dongjin) were sown on MS (Murashige and Skoog) media and incubated in a dark growth chamber for four days at 28° C. Seedlings were then transferred to a growth chamber with a light/dark cycle of 16 h light/8 h dark at 30° C. and grown to the two-week-old seedling. The seedlings were acclimatized in water for one day before being transferred to the Yoshida solution. They were then grown to three-week-old plants in Yoshida solution.

Three-week-old plants grown in Yoshida liquid media were fed with 10 mM randomly selected FAAs, including L-valine, leucine, and isoleucine (Sigma, USA) by dipping their roots into a corresponding FAA solution for 24 hrs. Plants pretreated with BCAA were harvested for amino acid analysis. Plants were then transferred to a 50 ml tube containing 30 mL of 21% PEG 8000 (Sigma) solution. PEG-induced visual symptoms such as leaf rolling and wilting were monitored by imaging plants at the indicated time points using an a5000 (Sony) camera. Air-drying-induced drought phenotypes of FAA pretreated NT plants were recorded through relative water content (RWC) and symptoms analysis. Calculation formula of RWC(%)=(FW−DW)/(TW−DW)×100. FW: fresh weight, DW: dry weight, TW: turgid weight.

4. RNA Extraction and Quantitative Real-Time PCR (qRT-PCR) Analysis
1) Rice

To investigate the spatiotemporal gene transcript patterns of OsBCAT2, total RNA samples were extracted from the roots, stem, leaf, and flowers from different developmental stages of rice plants using a Hybrid-R RNA purification kit (GeneAll, Korea) according to the manufacturer's instructions. To measure the expression levels of OsBCAT2, total RNA samples were isolated from the shoots and roots using a Hybrid-R RNA purification kit (GeneAll, Korea). The cDNAs of total and/or immunoprecipitated RNAs were synthesized using the REVERTAID First Strand cDNA Synthesis Kit with an oligo (dT) primer (Thermo Scientific, Waltham, MA, USA). 20 ng of cDNA was utilized as a template for qRT-PCR analysis. qRT-PCR was carried out using 2×qRT-PCR Pre-mix with 20×EvaGreen (SolGent, Korea) and ROX dye (Promega, Madison, WI, USA). The amplification reactions were carried out at 95° C. for 10 minutes, followed by 40 cycles at 95° C. for 30 seconds, 60° C. for 30 seconds, and 72° C. for 30 seconds in a 20 μL volume mix containing 1 μL EvaGreen Mix. The rice UBIQUITIN1 (Os06g0681400) were used as an internal standard.

TABLE 3
Primer information used in the present
invention

| Pur- | | Gene | Sequence (5′-3′) |
|---|---|---|---|
| pose | Gene ID | Name | (SEQ ID NO:) |
| Clone | Os03g0231600 | OsBCAT2 | F: |
| | | | TTGCTCCGTGGATCCATGGCT |
| | | | GCTGCTGCTGCTGC |
| | | | (SEQ ID NO: 5) |
| Clone | Os03g0231600 | OsBCAT2 | R: |
| | | | AAAGCGGCCGCAAATATCTAT |
| | | | TGCCACTGTCCATCC |
| | | | (SEQ ID NO: 6) |
| RT- | Os03g0231600 | OsBCAT2 | F: |
| PCR | | | GTGCACAAGACGTACCTGGA |
| | | | (SEQ ID NO: 7) |
| RT- | Os03g0231600 | OsBCAT2 | R: |
| PCR | | | GGAGACGAGCCGTTCTTCAA |
| | | | (SEQ ID NO: 8) |
| RT- | Os05g0586200 | OsJarl | F: |
| PCR | | | GCTTCCACAACTCCACACCT |
| | | | (SEQ ID NO: 9) |
| RT- | Os05g0586200 | OsJar1 | R: |
| PCR | | | CGCTTGATCGTTCCACGAAG |
| | | | (SEQ ID NO: 10) |
| RT- | Os05t0190500 | OsVSP2 | F: |
| PCR | | | GGGAACACTGCGACTCTACC |
| | | | (SEQ ID NO: 11) |
| RT- | Os05t0190500 | OsVSP2 | R: |
| PCR | | | GCCCAACAGGCTGGAGTAAT |
| | | | (SEQ ID NO: 12) |
| RT- | Os11t0605500 | OsACX2 | F: |
| PCR | | | CATGTGGTGGCCATGGGTAT |
| | | | (SEQ ID NO: 13) |
| RT- | Os11t0605500 | OsACX2 | R: |
| PCR | | | ATCTCCAGCAACCTGCTGAA |
| | | | (SEQ ID NO: 14) |
| RT- | Os02t0194700 | OsLOX1 | F: |
| PCR | | | CATGCCGTCCAAGATGCA |
| | | | (SEQ ID NO: 15) |
| RT- | Os02t0194700 | OsLOXI | R: |
| PCR | | | GGAGTGCGACGACAGGATGT |
| | | | (SEQ ID NO: 16) |
| RT- | Os05t0304600 | OsLOX7 | F: |
| PCR | | | CCGATATCTATGCCCATTGGA |
| | | | (SEQ ID NO: 17) |
| RT- | Os05t0304600 | OsLOX7 | R: |
| PCR | | | CCATCCCCCTCTTGATGAGA |
| | | | (SEQ ID NO: 18) |
| RT- | Os08t0509100 | OsLOX8 | F: |
| PCR | | | GCAGCTCAGCGAGATGCA |
| | | | (SEQ ID NO: 19) |
| RT- | Os08t0509100 | OsLOX8 | R: |
| PCR | | | CGTTGATCCGCATCGTGTAC |
| | | | (SEQ ID NO: 20) |
| RT- | Os01g0165000 | OsAPX2 | F: |
| PCR | | | TCCTACGCCGACTTCTACCA |
| | | | (SEQ ID NO: 21) |
| RT- | Os01g0165000 | OsAPX2 | R: |
| PCR | | | CGGCGTAATCCGCAAAGAAG |
| | | | (SEQ ID NO: 22) |
| RT- | Os06g0115400 | OsSOD1 | F: |
| PCR | | | CAGGTTGAGGGAGTCGTCAC |
| | | | (SEQ ID NO: 23) |
| RT- | Os06g0115400 | OsSODI | R: |
| PCR | | | GGTTGCCTCAGCTACACCTT |
| | | | (SEQ ID NO: 24) |
| RT- | Os06g0143000 | OsSOD2 | F: |
| PCR | | | GTGAAGGCTGTTGTTGTGCT |
| | | | (SEQ ID NO: 25) |
| RT- | Os06g0143000 | OsSOD2 | R: |
| PCR | | | GCCAGAGACACTTCCAGTCA |
| | | | (SEQ ID NO: 26) |
| RT- | Os06g0727200 | OsCAT | F: |
| PCR | | | TACTTCCCATCCCGCTACGA |
| | | | (SEQ ID NO: 27) |
| RT- | Os06g0727200 | OsCAT | R: |
| PCR | | | TCCTTACATGCTCGGCTTCG |
| | | | (SEQ ID NO: 28) |
| RT- | Os06g0681400 | OsUbi | F: |
| PCR | | | GCCAAGATCCAGGACAAGGA |
| | | | (SEQ ID NO: 29) |
| RT- | Os06g0681400 | OsUbi | R: |
| PCR | | | GCCATCCTCCAGCTGCTT |
| | | | (SEQ ID NO: 30) |

2) Soy Bean

To investigate the gene expression patterns of GmBCATs, total RNA samples were extracted, by using the Hybrid-R RNA Purification Kit according to the manufacturer's instructions, from roots, stems, and leaves of 4-week-old soybeans after drought, salt, or ABA treatments. To measure the expression levels of GmBCATs, total RNA was isolated from shoots and roots using the Hybrid-R RNA Purification Kit (GeneAll, Korea). cDNA from total and/or immunoprecipitated RNA was synthesized using the RevertAid™ First Strand cDNA Synthesis Kit with oligo (dT) primers, and 20 ng of cDNA were used as a template for qRT-PCR analysis. qRT-PCR was performed using 2×qRT-PCR Premix and 20×EvaGreen and ROX dyes (SolGent, Korea). The amplification reaction was performed by reacting a 20 μl volume mixture containing 1 μl Eva Green Mix at 95° C. for 10 minutes, followed by 40 cycles of 95° C. for 30 seconds, 60° C. for 30 seconds, and 72° C. for 30 seconds. Soybean GmACT1 was used as an internal standard.

TABLE 4
Information of primers used for determination
of expression level of GmBCATs

| Primer | Sequence (5′-3′) |
|---|---|
| name | (SEQ ID NO:) |
| GmAct1_F | CGCGTTTCGTTTGTGCAAGGT |
| | (SEQ ID NO: 36) |
| GmAct1_R | CAGTATGGCGTGGTCTCCCA |
| | (SEQ ID NO: 37) |
| GmBCAT1_F | AAGAACTGTGTCCTTTCCCAGT |
| | (SEQ ID NO: 38) |
| GmBCAT1_R | TAGGGTTGTGGCTCGGTAGA |
| | (SEQ ID NO: 39) |
| GmBCAT2_F | GCTTCTTCGGGCTGGTTCTAA |
| | (SEQ ID NO: 40) |
| GmBCAT2_R | ATACTCGTCATCACTGTGACTCC |
| | (SEQ ID NO: 41) |
| GmBCAT3_F | TGCAGCGTTTTGGGAACATC |
| | (SEQ ID NO: 42) |
| GmBCAT3_R | ACTCCCATCTTGTTTGCGGT |
| | (SEQ ID NO: 43) |
| GmBCAT4_F | AGCGCCGTCTACGGAAG |
| | (SEQ ID NO: 44) |
| GmBCAT4_R | ATGAGAGGAAACTGCTTGCGA |
| | (SEQ ID NO: 45) |
| GmBCAT5_F | GTTAGGCAACCGCAAAGACG |
| | (SEQ ID NO: 46) |
| GmBCAT5_R | CCTCGTTTCATGCGTTGAGC |
| | (SEQ ID NO: 47) |
| GmBCAT6_F | GGAACGTGCTGTATCAGTGGA |
| | (SEQ ID NO: 48) |
| GmBCAT6_R | GTGTTGCGCGTAATTTTGCTGA |
| | (SEQ ID NO: 49) |
| GmBCAT7_F | GGTTTGGCAGAGCTGGTTTG |
| | (SEQ ID NO: 50) |
| GmBCAT7_R | CTTTGGACCTGCTTCCCACT |
| | (SEQ ID NO: 51) |

5. Drought Stress Treatment and Stress Measurement at the Vegetative Stage For 4 days, osbcat2 and NT (Oryza sativa L. ssp. Japonica cv. Dongjin) seedlings were germinated on the Murashige-Skoog (MS) medium in a dark growth chamber at 28° C. Before the transplant, germinated plants were acclimated to light conditions for one day. The 5-day-old seedlings were transplanted into soil pots (4×4×6 cm, three plants per pot) within a container (59×38.5×15 cm) and grown for five weeks in greenhouse conditions at the automatically controlled glass-greenhouse condition (light/dark cycle of 16 h light/8 h dark at 30° C. ±3° C.). Drought treatment for observation or measurement was uniformly imposed by withholding water and re-watering at the vegetative phase of rice. Soil moisture was monitored during the test and measured by a soil moisture sensor (SM159, Delta-T Devices Cambridge, United Kingdom).

To measure the drought stress of osbcat2 and NT, we evaluated the efficiency of photosystems I and II through JIP test (Strasser, R. J., Tsimilli-Michael, M., Srivastava, A. (2004). Analysis of the Chlorophyll a Fluorescence Transient. In: Papageorgiou, G. C., Govindjee (eds) Chlorophyll a Fluorescence. Advances in Photosynthesis and Respiration, vol 19. Springer, Dordrecht.). These data were recorded using a Handy-PEA fluorimeter (Plant Efficiency Analyzer Hansatech Instruments, UK) in dark conditions to impose optimal dark adaptation (at least 20 min). Ten leaves were collected and calculated using the Handy PEA software (version 1.31) and analyzed according to the calculation formula of the JIP test.

### 6. Analysis of Agronomic Traits of Rice in a Rice Paddy Field

To evaluate the agronomic characteristics and traits of osbcat2 and NT rice plants, five independent Tl homozygous mutants and NT (Oryza sativa L. ssp. Japonica cv. Dongjin) plants were cultivated in the paddy field at the Kyungpook National University, Gunwi (128: 34E/36: 15N), Korea. We divided the paddy field into three sections to minimize the influence of microclimate, and each plot was planted with ten plants per line. The experimental environment was created for agronomic evaluation of osbcat2 and NT plants under dehydration conditions. Plants were grown in an artificially created reservoir with a rain-off shelter. Water deficit conditions is imposed to plants before and after the heading phase by controlling water levels. When phenotypes caused by drought stress were observed in NT, re-irrigation was performed for recovery. The yield components of 10 plants per line from three different plots for the drought field conditions were measured.

### 7. DAB and NBT Staining

For DAB staining, 50 mg 3,3′-Diaminobenzidine (Sigma) was dissolved in 45 ml H₂O₂ then the pH was adjusted to 3.8 with 0.1 N HCl. To this solution, 25 μL of Tween20 (0.05% v/v) and 2.5ml of Na₂HPO₄ (200 mM) were added and filled with H₂O₂ to make a total volume of 50 ml. Leaf sections of accurately 2.5 cm in length were cut and soaked in a 1% DAB solution. After 30 min vacuum infiltrating, the immersed leaves were incubated in the dark for 20 h at room temperature. And then, the leaves were bleached by bathing and boiling ethanol until the brown spots appeared clearly. The area of brown spots represents the DAB reaction degree to H₂O₂.

In order to detect superoxide accumulation using a 0.2% solution of NBT in a 10 mM potassium phosphate buffer (pH 7.7), roughly 2.5 cm-long leaf pieces were cut. After 15 minutes of vacuum infiltration, the submerged leaves were incubated at room temperature for overnight. After incubation, the leaves were fixed and clarified in alcoholic lacto-phenol (2:1:1, % ethanol:lactic acid:phenol) at 65° C. for 30 minutes, rinsing with 50% ethanol, and then rinsing with water. In leaves, a blue precipitate form when NBT reacts with superoxide.

### 8. Subcellular Localization of Soybean GmBCATs Using Rice Protoplast

Protoplasts were isolated and prepared from rice (Oryza sativa cv. Dongjin) seedlings. Specifically, rice seedlings were grown on MS medium at 28° C. under dark conditions for 10 days and then exposed to light conditions for 10 h. Leaf sheaths of 50 rice seedlings were cut into 0.51 mm pieces on a glass plate using a sharp knife. The cut pieces were collected and transferred to a 0.6 M mannitol solution and cultured under dark conditions at 25° C. for 30 min. Afterwards, the mannitol solution was removed, and the fragments were placed in a digestion solution (1.5% Cellulase R-10 (Yakult, Japan), 0.75% Macerozyme R-10 (Yakult, Japan), 0.6 M mannitol, 10 mM MES (pH 5.7), 0.1% BSA, 10 mM CaCl₂, 5 mM B-mercaptoethanol) for cell wall degradation. The cell walls were digested under gentle shaking (30 to 40 rpm) at 28° C. in the dark for 4 to 5 hours. Vacuum infiltration was intermittently performed for 20 min every 1 hour using a desiccator during the digestion process. After the digestion was completed, the protoplasts were collected in a 50 mL tube containing 20 mL of W5 (154 mM NaCl, 125 mM CaCl₂, 5 mM KCl, 2 mM MES (pH 5.7)) solution.

The protoplast suspension was filtered twice using 70 μm and 40 μm nylon mesh (Falcon, USA) to remove debris. The filtered suspension was centrifuged at 320 g to obtain a protoplast pellet, which was then re-suspended in MMG solution (0.6 M mannitol, 15 mM MgCl₂, 4 mM MES (pH 5.7)).

The protoplast concentration was measured using a microscope and a hemocytometer (Marienfeld) and adjusted to 5.0×10⁷ protoplasts/mL. Fifty μL of protoplasts 2.0×10⁶ cells) were mixed with 15 μL of pMito-RFP mitochondrial marker and GmBCATs-GFP plasmid, and 130 μL of 40% PEG 4000 solution were added. The mixture was incubated in the dark at 28° C. for 15 min. After the incubation, 1 mL of W5solution was added to the mixture, and the protoplasts were collected by centrifugation at 300 g for 2 minutes. The collected protoplasts were re-suspended in the incubation solution and cultured for 10 hours. The protoplasts were recovered by centrifugation at 300 g for 2 minutes, and the location of the fluorescent protein was analyzed using a confocal laser microscope (LEICA, SP8 confocal microscope).

### 9. Drought Stress Treatment and Yield Analysis of Soybean

Soybean was sown in a pot (×4×6 cm, 1 plant per pot) in a container (59×38.5×15 cm) and seedling was grown for 2 weeks under automatically controlled glasshouse conditions (30° C.±3° C., 16 h light/8 h dark), then transferred to larger pots for cultivation. Drought treatment for observation or measurement was uniformly implemented by stopping water supply during the soybean cultivation stage followed by re-watering. Soil moisture was monitored during the test and measured by a soil moisture sensor (SM159, Delta-T Devices, UK).

To measure drought stress in Gmbcat1/2 and NT, survival rate analysis was conducted after drought treatment. After sufficiently watering 8-week-old R1 stage plants, water supply was withheld for 5 days in a greenhouse. Water was then resupplied, and the plant survival rate was analyzed after 5 days. On the other hand, for the analysis of agricultural traits after drought treatment, NT and Gmbcat1/2 plants were grown under normal water supply conditions or drought treatment, which has been carried out twice in the R1 stage (water supply withheld for 3 days), conditions. The final soybean yield per plant was measured in weight to analyze the agricultural productivity (more than 50 plants were analyzed for each genotype). Statistical significance was then analyzed using ANOVA analysis (**P<0.01).

### 10. Quantification of BCAA (Branched-Chain Amino Acid)

Leaves of 6-week-old NT and Gmbcat1/2 plants grown in a greenhouse were sampled, ground in liquid nitrogen, and extracted with an extraction solution. Amino acids were then quantified using HPLC equipment equipped with a VDSpher 100 C18-E column (4.6 mm×150 mm, 3.5 μm/VDS, Optilab, USA) and a 1260 FLD FL detector (Agilent, USA).

### Example 1. OsBCAT2 is a Drought-Responsive Mitochondrial BCAA Aminotransferase

For 12 genes including BCATs of Arabidopsis thaliana (AtBCATs) and BCATs of tomato (SIBCATs) that are taken as a subject, multiple amino acid sequence alignment was carried out. As a result of the phylogenetic analysis, very high homology was found among BCAT genes of Arabidopsis thaliana, tomato, and rice, including AtBCAT2 and SIBCAT2 that are known to be present in mitochondria. Amino acid sequence homology of BCAA aminotransferase domain among them was more than 80% and it was recognized that difference of the amino acids sequence was found only in the signal peptide sequence region at the N terminus which determined the subcellular localization of the protein.

In order to find the drought-induced branched-chain amino acid aminotransferase genes, we used RNA-sequencing (RNA-seq) in a previous study (Chung et al. 2016 BMC Genomics. 17:563), 5 drought-responsive (2 up and 3 down-regulated, respectively, with a log2 ratio ≥2.0 and ≤−2.0) BCAA transferase-related gene were identified. Among these, OsBCAT2 was up-regulated by drought stress.

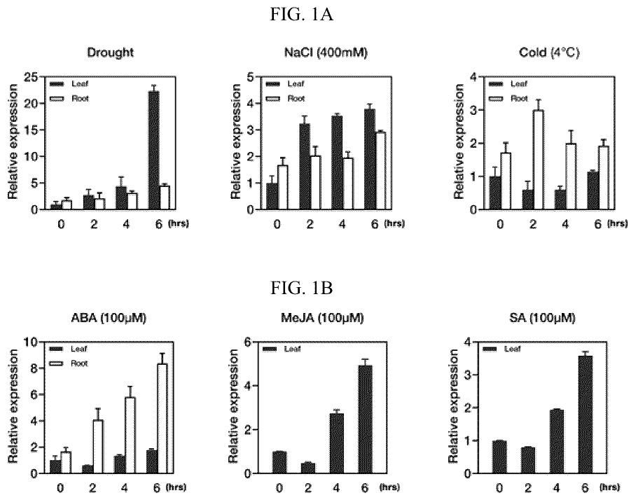

To confirm the transcription levels in OsBCAT2 in various abiotic conditions, 2-week-old rice seedlings were exposed to drought, high salt, abscisic acid (ABA), and low temperature. These analyses show that OsBCAT2 transcript level was strongly induced within 6 hr of exposure to drought stress in leaves than in other abiotic stress (FIG. 1A). These results are similar to RNA-seq data. Moreover, specifically in roots, the transcript level significantly increased after phytohormone (ABA, JA, SA) treatment (FIG. 1B). OsBCAT2 might have multiple enzymatic roles in response to abiotic stress.

### Example 2. OsBCAT2 is Involved in BCAA Catabolism

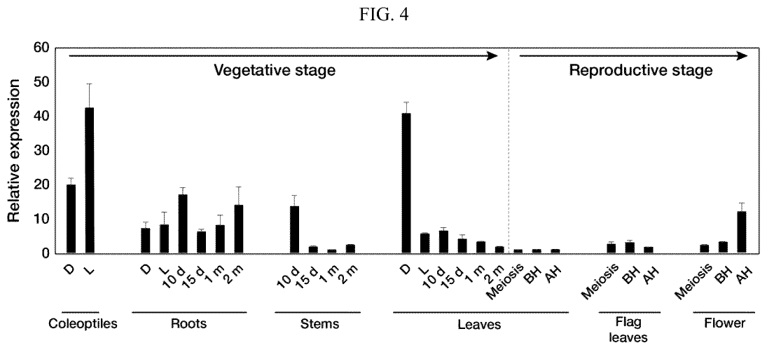

Amino acids are involved in most metabolic pathways and known to regulate various developmental processes in plants. We performed the transcription analysis of OsBCAT2 by qRT-PCR. The expression level was checked in various tissues at different developmental stages, starting from vegetative to developmental stage. These data showed that OsBCAT2 transcripts were up-regulated from all the rice tissues. OsBCAT2 showed higher expression during the developmental stage than at the vegetative stage, specifically in leaves in dark conditions (FIG. 4). This transcript pattern is similar to that of the gene that is related to BCAA catabolismin Arabidopsis.

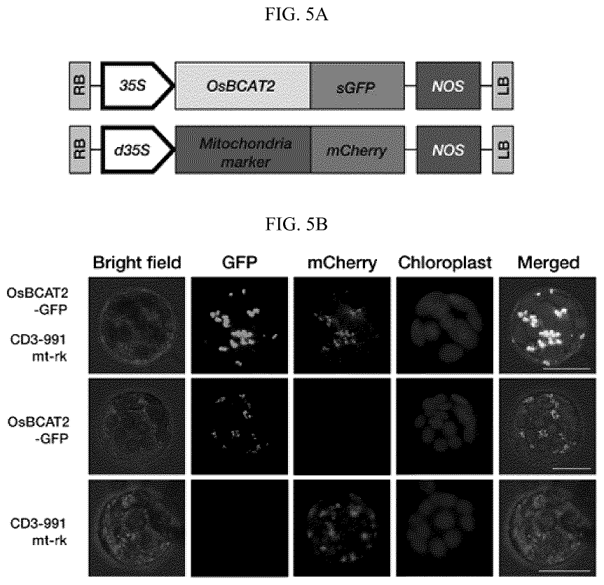

It is known that major enzymes involved in biosynthesis and degradation of BCAA are present in chloroplast and mitochondria, respectively. Accordingly, by expressing OsBCAT2-GFP fusion protein in rice protoplast, the inventors of the present invention investigated the subcellular localization of OsBCAT2 in order to speculate whether OsBCAT2 is involved in the synthesis or degradation of BCAA. As a result of confocal laser scanning microscopy analysis, the fluorescent signal of OsBCAT2-GFP protein was observed only from mitochondria. In addition, as a result of subcellular localization analysis, it was found that the expression of OsBCAT2 protein is similar to the expression of mitochondria marker (FIGS. 5A and 5B). These results suggest that, in mitochondria, OsBCAT2 is involved in catabolizing BCAA.

### Example 3. Loss of OsBCAT2 Confers Enhanced Tolerance to Drought

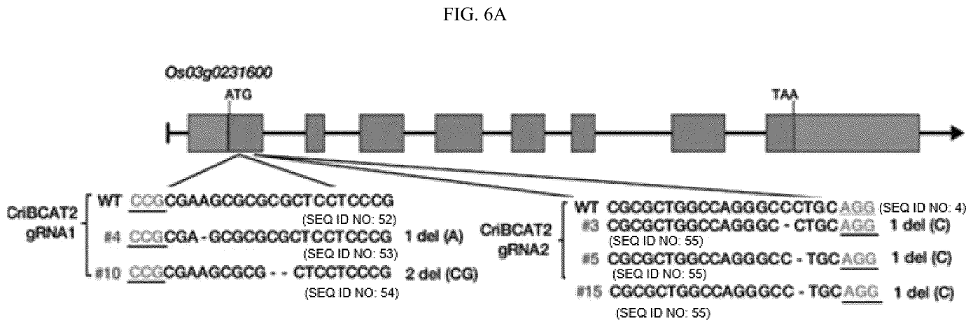

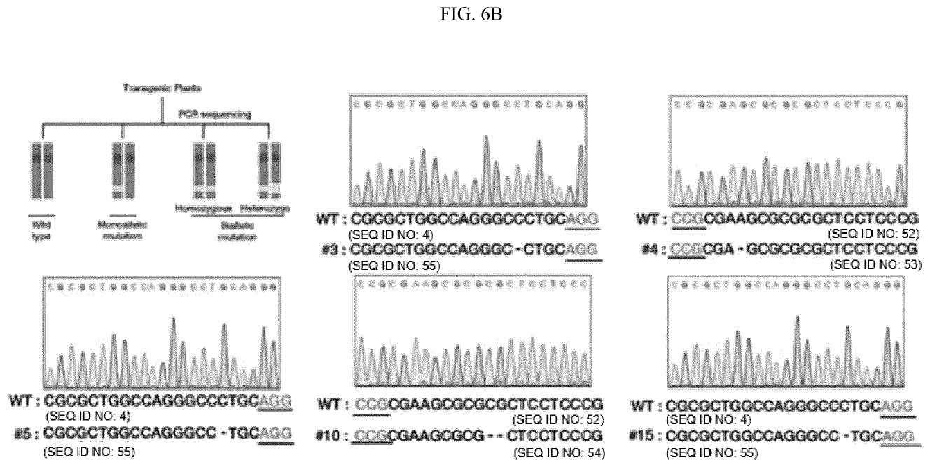

A previous study about OsDIAT overexpressing transgenic plants showed that increasing BCAA content in leaf tissue confers enhanced tolerance to drought stress (U.S. Pat. No. 11,046,970B2). To investigate the biological functions of osbcat2 mutants under drought stress, we designed two types of single guide RNA (sgRNA) for CRISPR-Cas9 systems technology to achieve knockout mutants in OsBCAT2 gene and obtained homozygous five individual mutants (#3, #4, #5, #10, #15) (FIG. 6A). Both sgRNAs were designed in the first exon, gRNA1 mutants (#4, #10) had 1-base, 2-base deletion and gRNA2 mutants (#3, #5, #15) had 1-base deletion in different DNA sequences, respectively. Sequencing analysis revealed mutation patterns in transgenic plants, including frameshift and premature stop codon (FIG. 6B).

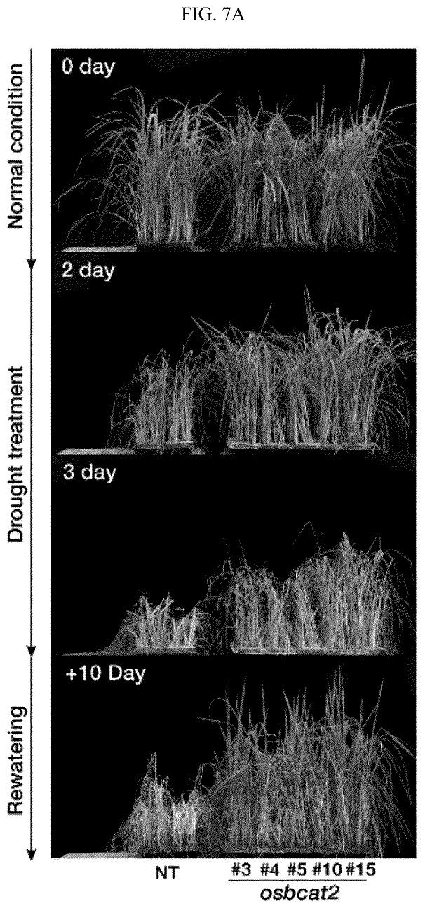

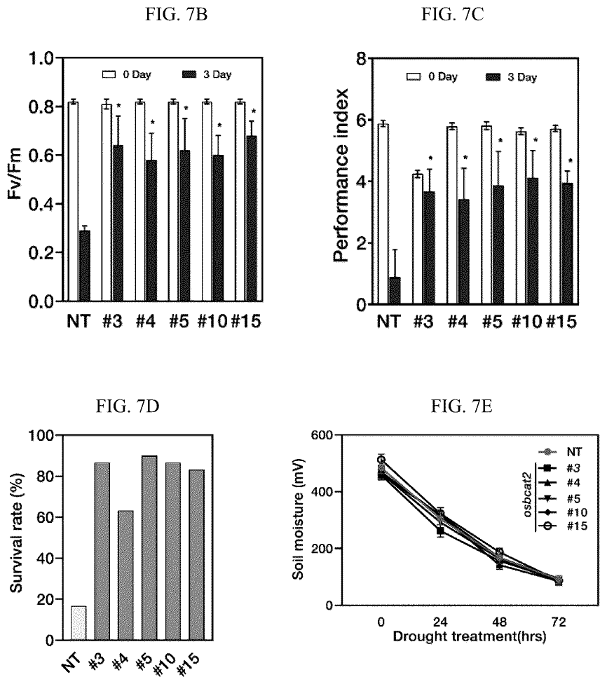

We conducted a drought tolerance test on osbcat2 mutants to examine their response to drought stress and biological roles. In the greenhouse, 4-week-old plants were subjected to drought stress by withholding water for several days (FIG. 7A). Soil moisture indicated a constantly decreasing for 3 days, indicating that the drought stress was uniformly applied to plants, 1 day after drought treatments, drought-induced damage was displayed in NT plants (FIG. 7E). 2 to 3 days after exposure to drought conditions, most NT plants were induced unrecoverable symptoms from drought stress, including leaf wilting, rolling, and chlorosis. Whereas all osbcat2 mutant lines exhibited resistance to the drought stress and far less drought-induced symptoms after 3 days of drought exposure, and they had a higher recovery rate than NT plant after re-watering. To further verify the drought tolerance of osbcat2 mutants, we determined the Maximum quantum efficiency of PS II system (Fv/Fm) values and photosynthetic performance index (PI) of PS I and PS II, which represent photochemical efficiency under abiotic stress. The Fv/Fm value of NT plants significantly decreased 3 days after stress exposure. On the other hand, osbcat2 mutants showed a slighter decrease in Fv/Fm value (FIG. 7B). Also, the Performance Index value also dramatically decreased in NT plants, whereas the PI value of osbcat2 was well controlled during drought exposure conditions (FIG. 7C). These results indicated that osbcat 2 mutants had higher drought tolerance than NT plants.

### Example 4. BCAA Levels are Significantly Accumulated in Osbcat2 plants d

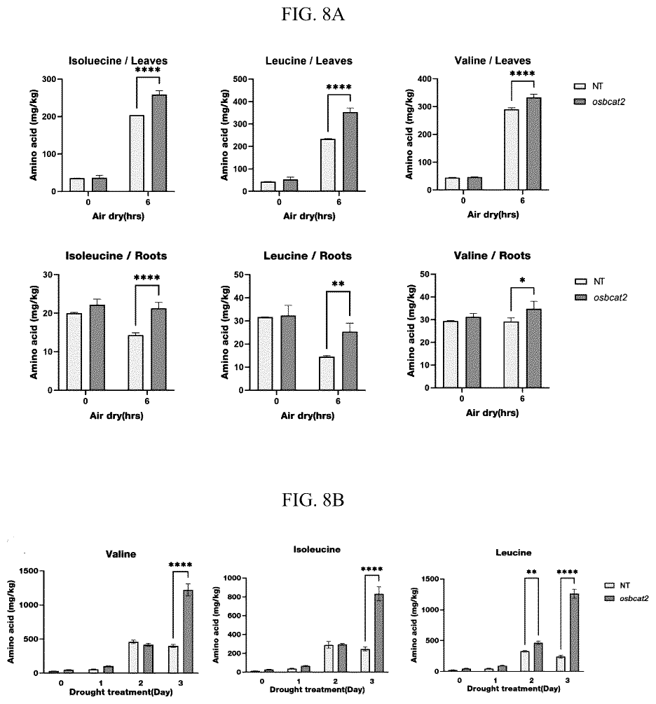

It was reported that Arabidopsis plants increased BCAA in response to drought and osmotic stress. We evaluated whether BCAA levels in NT and osbcat2 mutant are different under drought conditions. For High-performance liquid chromatography (HPLC) analysis, we exposed them (6-week-old plants) to drought stress conditions by air drying (6 hr). We measured the branched-chain amino acid content under normal and drought conditions. Compared to normal conditions samples, BCAA levels significantly increased under drought stress conditions. Specifically, osbcat2 mutants accumulated much higher BCAA than NT plants (FIG. 8A). To further validate amino acid changes according to the drought treatment period, we exposed them to soil drought stress conditions for three days. Leucine content in osbcat2 mutants was increased during first two days. Further, on three days, BCAA levels rapidly increased. On the other hand, BCAA content of NT plants was induced during first two days but decreased on the third day. Total BCAA content of osbcat2 was substantially increased compared to NT plants under drought conditions (FIG. 8B).

In order to further confirm whether the OsBCAT2 gene is involved in BCAA catabolism, we analyzed BCAA content in dried rice seed. The result showed that BCAA content exponentially increased in osbcat2 mutants than that in NT plants. Overall, these results indicate that drought stress leads to BCAA accumulation in rice plants and the expression of OsBCAT2. involving in BCAA catabolism, is induced by drought stress.

### Example 5. The External Supply of Branched-Chain Amino Acids Enhances Osmotic Shock Tolerance in Rice

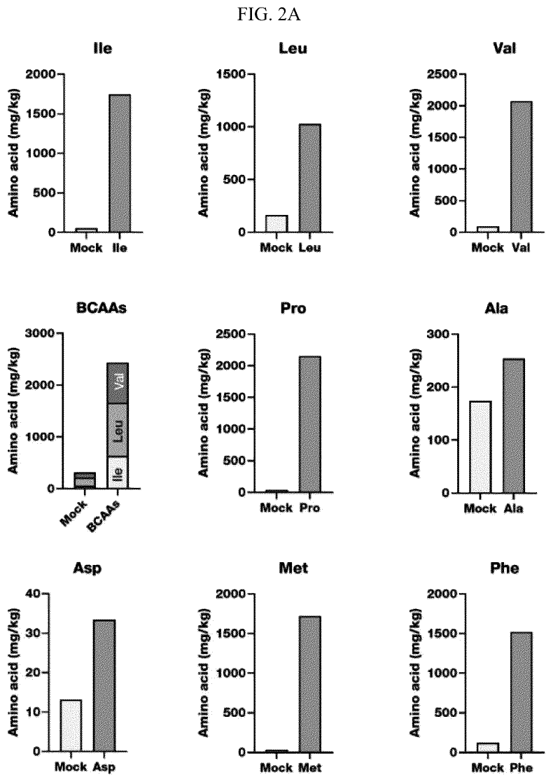

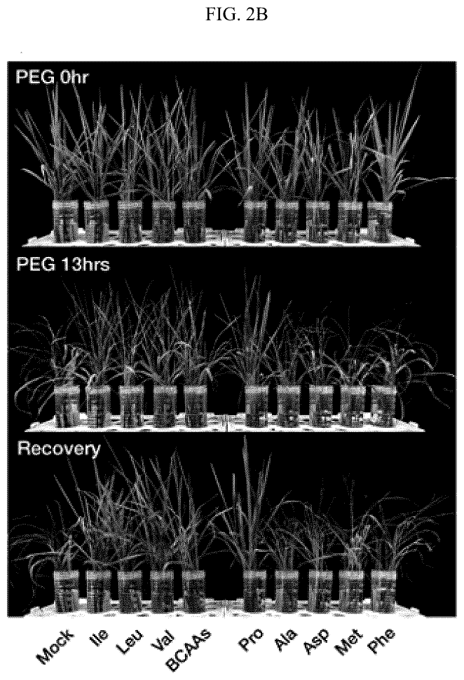

To determine the effect of amino acid pre-treatment in osmotic stress tolerance, various amino acids (i.e., proline, alanine, aspartic acid, methionine, isoleucine, leucine, valine, and BCAA compounds) were applied to NT rice for 24 hours before osmotic stress treatment. The pre-treated plants were examined by HPLC analysis. The result showed that the internal amino acid level has been significantly elevated by external application (FIG. 2A). Then, the pre-treated plants were applied with 21% PEG 8000 for simulating the osmotic stress. After the PEG treatment for 13 hours, symptoms of osmotic stress, including leaf curling and wilting, were observed from most NT plants pre-treated with amino acids, except BCAA and proline. Level of the symptoms was the strongest in Mock group followed by Ala, Asp, Met, Phe, BCAA, and Pro pre-treated group (FIG. 2B). It was also shown that the exogenous BCAA pre-treatment can promote the regeneration of plants damaged by PEG treatment after re-watering and also alleviate the symptoms of osmotic shock.

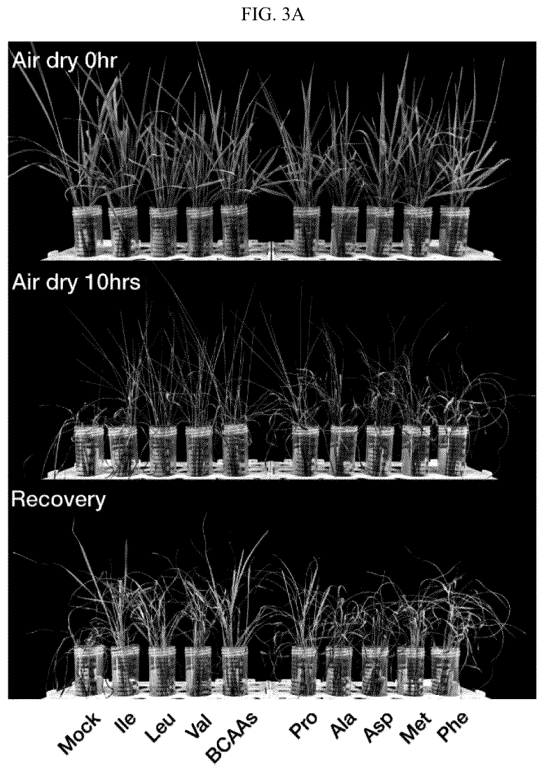

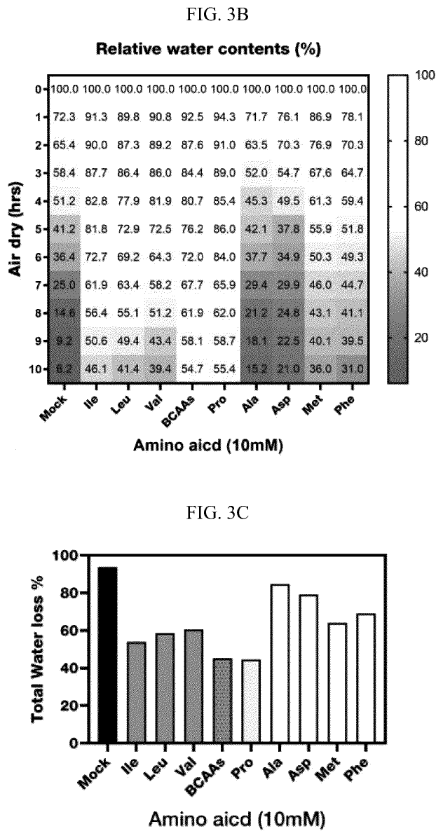

Furthermore, to determine the effect of amino acid accumulation in rice under severe osmotic stress, an air-drying test was carried out. First, a pre-treatment with amino acids was carried out for 24 hours in the same manner as the treatment method described in the above. After the air-drying treatment for 10 hours, it appears that significant damages are caused by drought stress in all plants. However, the plant treated with BCAA or proline showed less damage compared to the plant groups which have been pre-treated with other amino acids (FIG. 3A). The plant with mock treatment showed unrecoverable symptoms even after re-watering, while plant pre-treated with BCAA or proline showed recovery after re-watering. Based on these results, it was found that a pre-treatment with individual BCAA, a treatment with combination of BCAAs, and proline treatment can provide tolerance to drought stress, which has been caused by air drying. It was recognized that, similar to the PEG test described above, the BCAA and proline treatment groups can have recovery through a recovery process.

In order to further investigate the effect of supplying exogenous BCAA, level of moisture loss caused by air drying was monitored every hour. The results showed that the moisture loss was reduced by pre-treatment of any amino acid compared to that of plant with mock treatment (FIG. 3B). In particular, the pre-treatment with 3 individual BCAAs, combination of those BCAAs, or proline showed much better property of maintaining the moisture content compared to plants treated with Ala, Asp, Met, and Phe (FIG. 3C). In summary, BCAA accumulation in rice can provide the tolerance to osmotic stress.

### Example 6. Genome Editing of OsBCAT2 Improves Agronomic Traits Under Field Drought Conditions

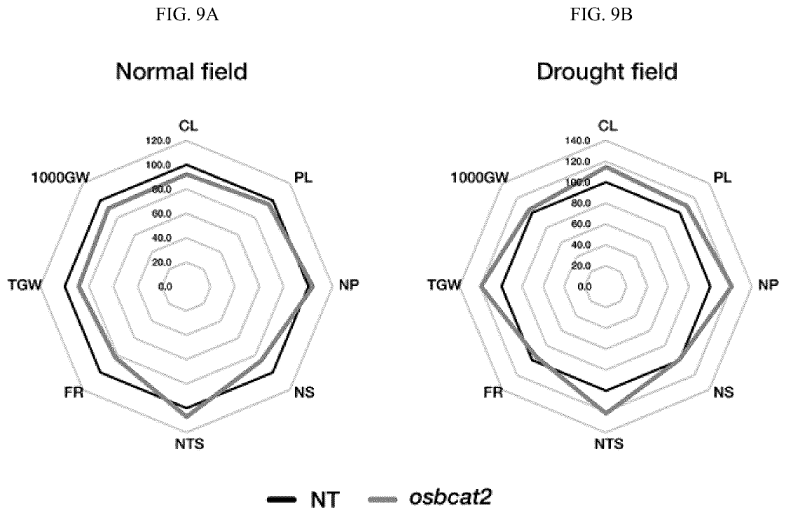

The above study revealed that loss of function osbcat2 mutant confers drought tolerance. To evaluate the yield performance of osbcat2 mutants under drought stress conditions, osbcat2 and NT plants were grown in the field conditions with rain-off shelters at Gunwi (128:34E/36:15N), Korea, for 100 days. Then, the sequential drought stress at the flowering and milk stage were applied by halting irrigation and drying 2-weeks. Under normal conditions, five independent osbcat2 were slightly lower than NT in the number of spikelets (NS) and Filling rate (FR). On the other hand, under drought conditions, osbcat2 significantly improved number of total seed (NTS) and total grain weight (TGW) (FIG. 9A and Table 5) compared to NT.

TABLE 5
The Agronomic trait of osbcat2 rice plants grown under normal and drought stress conditions

| | Culm | Panicle | No. of | No. of | No. of total | Filling | Total grain | 1000 grain |
|---|---|---|---|---|---|---|---|---|
| | length | length | panicles | spikelets | spikelets | rate | weight | weight |
| Constructs | (cm) | (cm) | (/hill) | (/panicle) | (/hill) | (%) | (g) | (g) |
| Normal | | | | | | | | |
| NT | 81.45 | 21.38 | 12.10 | 99.57 | 1219.20 | 82.18 | 24.25 | 24.06 |
| osbcat2 | 75.03 | 20.49 | 12.56 | 86.08 | 1310.25 | 69.15 | 20.99 | 21.82 |
| % Δ | −7.89 | −4.14 | 3.80 | −13.55 | 7.47 | −15.86 | −13.43 | −9.31 |
| p val | 0.042 | 0.098 | 0.284 | 0.004 | 0.216 | 0.002 | 0.089 | 0.076 |
| Drought | | | | | | | | |
| NT | 62.54 | 14.25 | 9.26 | 72.17 | 862.49 | 51.74 | 11.06 | 18.99 |
| osbcat2 | 70.16 | 15.57 | 11.14 | 72.81 | 1043.32 | 49.56 | 12.89 | 19.58 |
| % Δ | 12.18 | 9.26 | 20.30 | 0.89 | 20.97 | −4.21 | 16.55 | 3.11 |
| p val | 0.056 | 0.129 | 0.044 | 0.370 | 0.086 | 0.084 | 0.023 | 0.519 |

### Example 7. JA Induced Antioxidant Gene Meditates Drought Tolerance of Osbcat2 Mutants

Proline is one of the best characterized amino acids involving in drought tolerance in plants, and the mechanism of abiotic stress responses by proline has been studied well. However, the tolerance mechanism against abiotic stress by BCAA has been poorly understood so far.

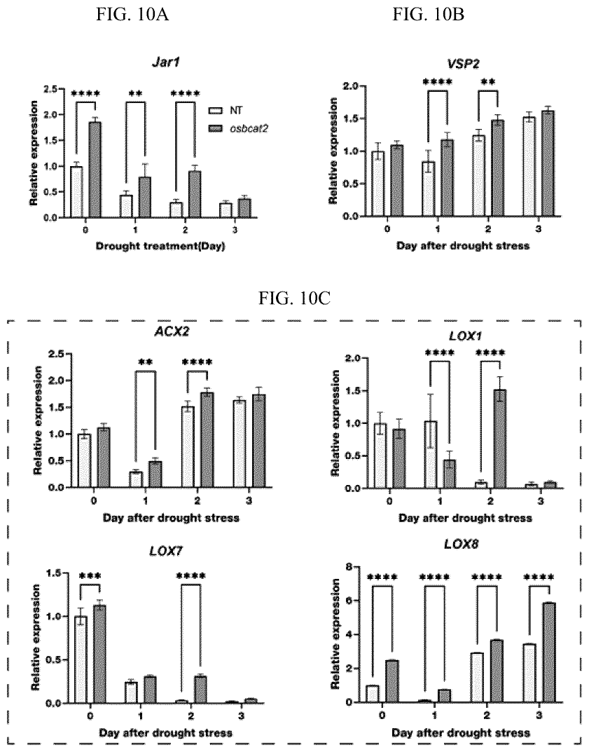

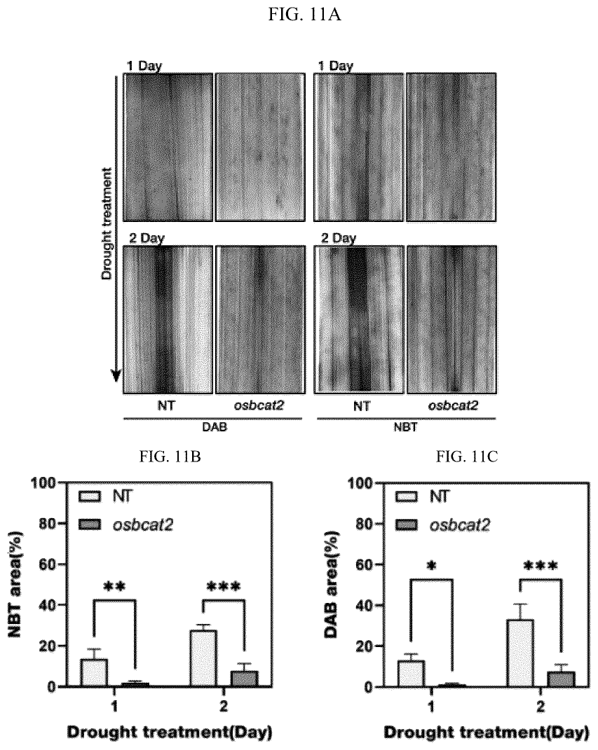

It has been well documented that biological active form of JA is JA-Ile, conjugated form with isoleucine in plants, and it is mediated by JASMONATE RESISTANT1 (JARI). To identify the function of the OsBCAT2 in JA signaling, we checked the transcription level of rice JARI under drought stress. JAR1 expression level was significantly higher in osbcat2 than in NT plants (FIG. 10A). Also, we confirm that expression level of JA-responsive genes enhanced in osbcat2 compared to NT plants (FIGS. 10B and 10C), suggesting that OsBCAT2 mutation promotes JA signaling.

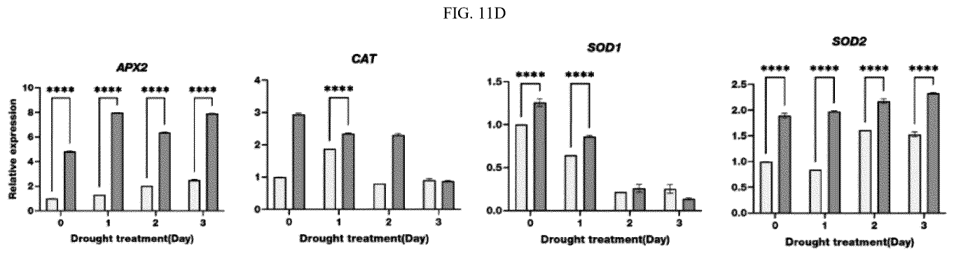

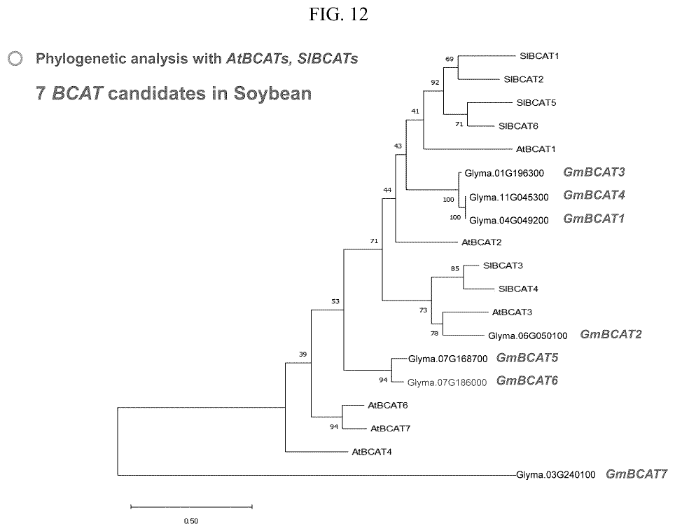

Expression of JA responsive antioxidant genes, such as APX, SOD, and POD, was highly up-regulated in osbcat2 (FIG. 11D). Then we conducted hydrogen peroxide (H₂O₂) and superoxide anion (O₂⁻) analysis in osbcat2 mutants to check ROS (Reactive Oxygen Species) accumulation. DAB and NBT staining, which provokes irreparable brown and blue products with reacting H₂O₂ and O₂-respectively, showed that osbcat2 mutants had less accumulated H₂O₂ than NT in response to drought stress (FIGS. 11A to 11D). Summarizing these results, drought stress-induced ROS was well controlled by JA-induced antioxidant genes in osbcat2, which confers drought tolerance.

### Example 8. GMBACT1 and GMBACT2 are Drought-Responsive BCAA Aminotransferase

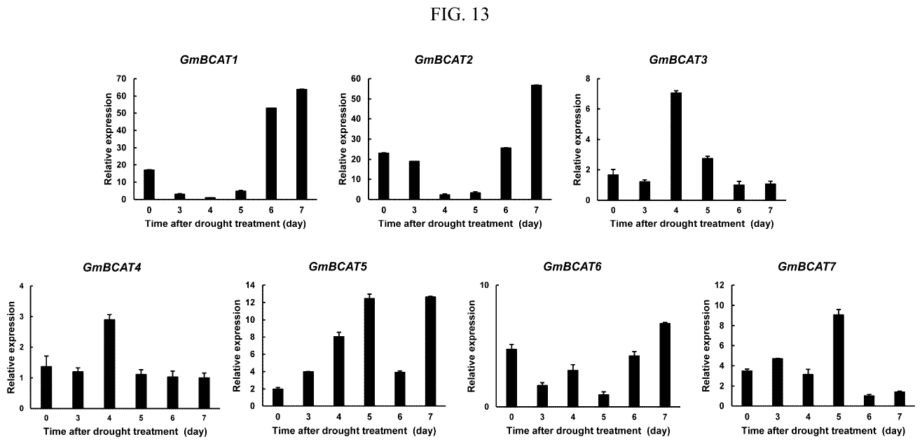

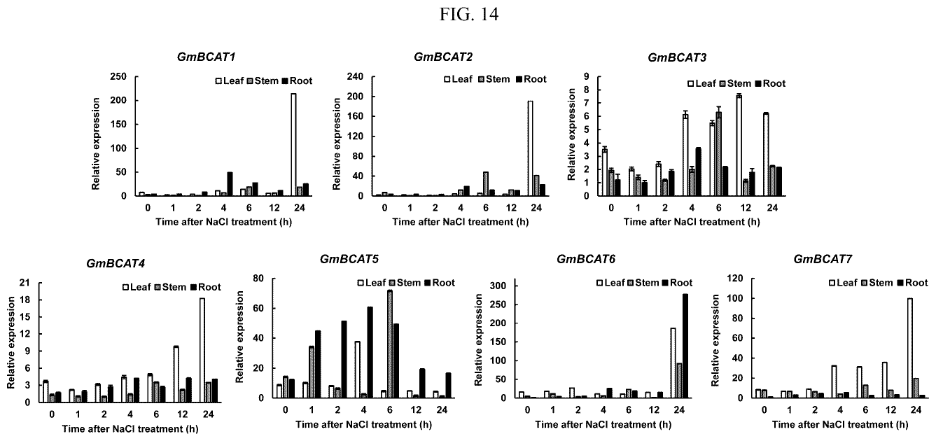

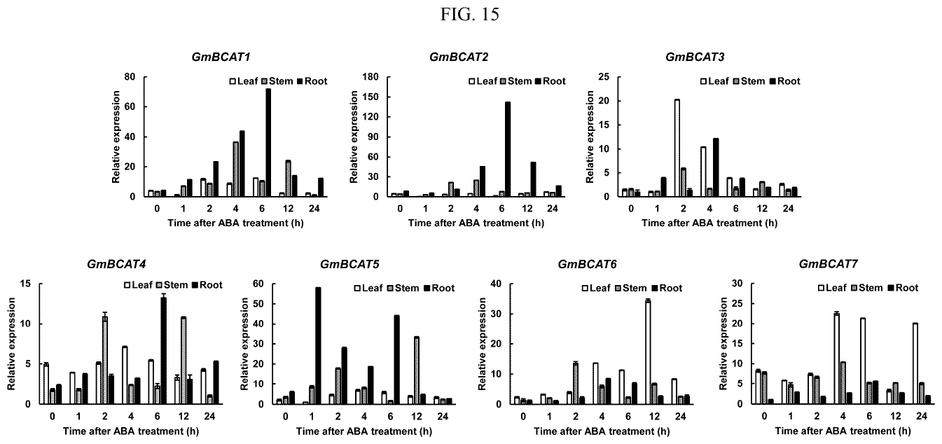

To determine the transcript levels of GmBCATs under various abiotic stress conditions, 4-week-old soybeans were treated with drought, high salt, or ABA, and the transcript levels were analyzed by RT-qPCR. As a result, the transcript levels of GmBCAT1 and GmBCAT2 were more highly induced in leaves exposed to drought stress after 6 days compared to other abiotic stresses (FIG. 13), and the expression was also significantly enhanced in leaves after 24 hours of salt treatment (FIG. 14). In addition, the transcript levels were significantly increased especially in roots after treatment with the plant hormone ABA (FIG. 15). It is thus believed that GmBCAT1 and GmBCAT2 may play various enzymatic roles in response to abiotic stresses.

### Example 9. GmBCAT1 and GmBCAT2 are BCAT involved in BCAA Catabolism

Amino acids are known to be involved in most metabolic pathways and regulate various developmental processes of plants. The present inventors performed transcriptional analysis of GmBCAT1 and GmBCAT2 by RT-qPCR. The analysis results showed that GmBCAT1 and GmBCAT2 transcripts were significantly increased in response to drought and salt stress in leaves (FIG. 14), and increased in response to ABA in roots (FIG. 15). These transcript patterns are similar to those of genes that are related to BCAA catabolismin rice and Arabidopsis thaliana.

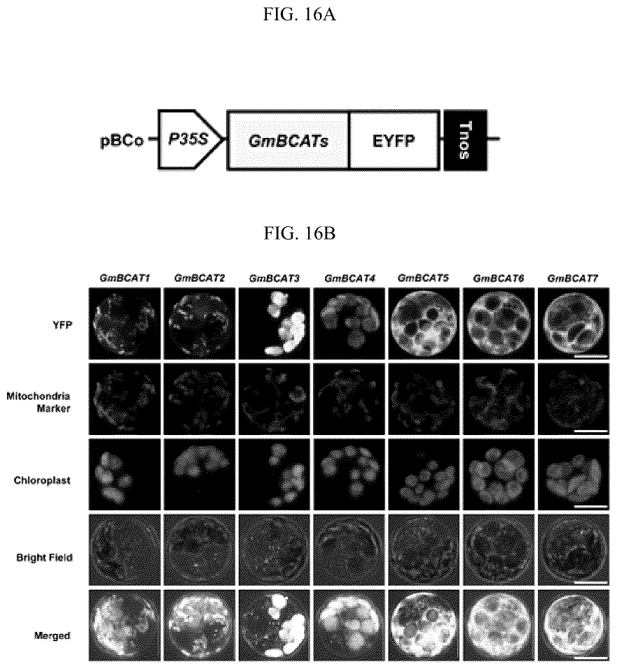

The major enzymes involved in BCAA biosynthesis and degradation are known to be located in chloroplasts and mitochondria, respectively. Therefore, the present inventors investigated whether GmBCAT1 and GmBCAT2 are involved in BCAA synthesis or degradation using GmBCAT1-GFP and GmBCAT2-GFP fusion proteins expressed in rice protoplasts. Confocal laser scanning microscopy analysis showed that fluorescence signals due to GmBCAT1-GFP and GmBCAT2-GFP proteins were observed only in mitochondria. In addition, subcellular localization analysis revealed that GmBCAT2 protein expression is similar to the expression of mitochondrial markers (FIGS. 16A and 16B). These results suggest that GmBCAT1 and GmBCAT2 can perform BCAA catabolismin mitochondria. In addition, when compared with the previous results obtained from rice OsBCAT2, it suggests that, in the case of BCATs located in mitochondria, even BCATs present in different plant species can act on BCAA degradation.

### Example 10. Loss of Function of GmBCAT1 and GmBCAT2 Enhances Drought Resistance

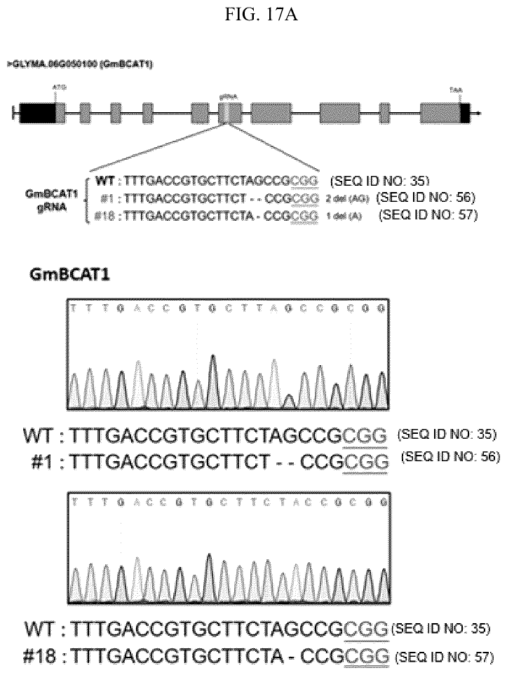

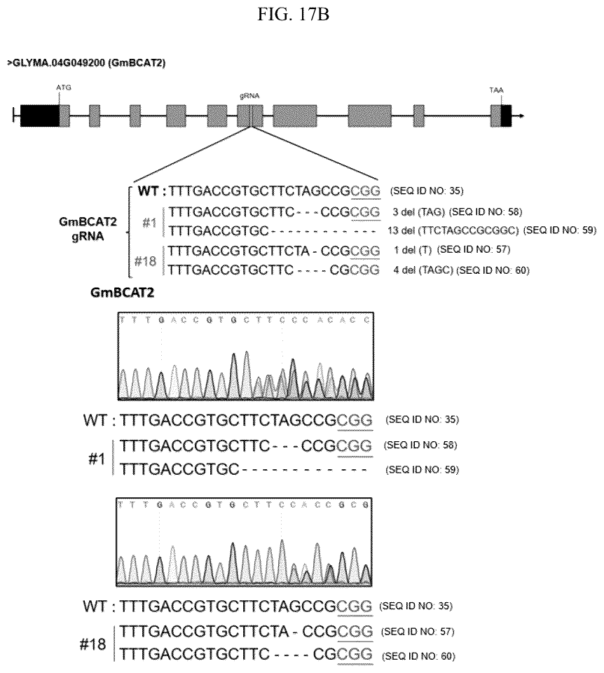

Previous studies on Osbcat2 mutants have shown that increasing BCAA content in leaf tissues enhances drought stress tolerance. To investigate the biological function of Gmbcat1/2 mutants in drought stress, one type of single guide RNA (sgRNA) for CRISPR-Cas9 system technology was designed to achieve knockout mutations in GmBCAT1 and GmBCAT2 genes, and two individual homozygous mutants (#1, #18) were obtained (FIGS. 17A and 17B). The sgRNAs were designed to target the common sixth exon of −GmBCAT1 and GmBCAT2, and mutant #1 had a 2 bp deletion of BCAT1 and 3 bp and 13 bp deletions of BCAT2. In addition, mutant #18 had a 1 bp deletion of BCAT1, and 1 bp and 4 bp deletions of BCAT2. As a result of sequencing analysis, mutation patterns in gene-modified plants, including frame shifts and premature stop codons, were found (FIGS. 17A and 17B).

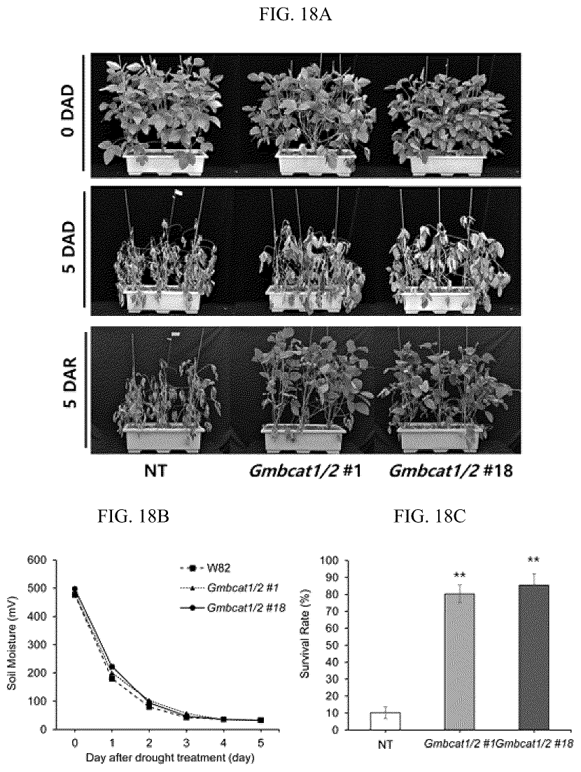

Drought tolerance test was performed on Gmbcat1/2 mutants to investigate their response to drought stress and their biological role. In the greenhouse, 5-week-old plants were subjected to drought stress by not receiving any water for several days (FIG. 18A). Soil moisture decreased continuously for 5 days, indicating that the plants were subjected to drought stress continuously and uniformly. After 5 days of drought stress, NT plants showed drought damage (FIGS. 18A and 18B). After 2 to 3 days of exposure to drought conditions, most NT plants showed irreversible symptoms due to drought stress, such as leaf wilting, curling, and yellowness. On the other hand, all Gmbcat1/2 mutants showed drought tolerance after 5 days of drought exposure, showed significantly fewer drought-induced symptoms, and had a higher recovery rate than NT plants after re-watering (FIG. 18C). These results indicated that Gmbcat1/2 mutants had higher drought tolerance than NT plants.

### Example 11. Genome Editing of Gmbcat1/2 Caused Higher Yield Under Greenhouse Conditions

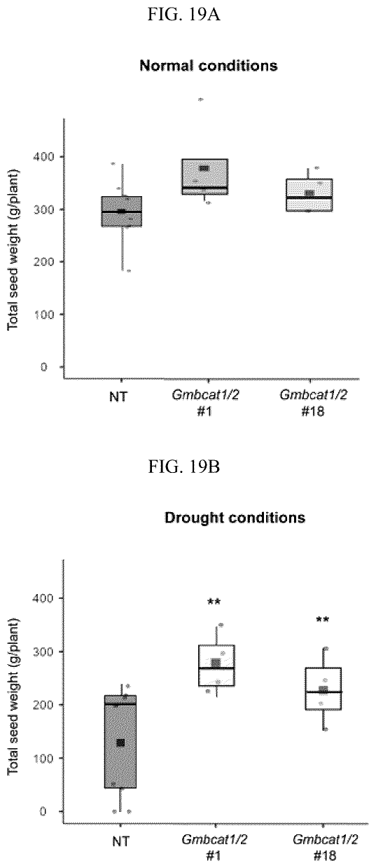

The above results indicated that the mutants lacking the function1/2 mutant provide drought tolerance. Therefore, to evaluate whether the Gmbcat1/2 mutant could affect the total grain yield under drought stress conditions, Gmbcat1/2 and NT plants were grown under greenhouse conditions for 200 DAS (days after sowing) from transplanting to maturity. Then, drought stress was applied twice consecutively during flowering and maturation periods. Under normal conditions, two independent Gmbcat1/2 strains did not differ from NT in total grain weight. On the other hand, under drought conditions, Gmbcat1/2 significantly improved the total grain weight by more than 30% compared to NT (FIGS. 19A and 19B). These results demonstrate that genome editing of Gmbcat1/2 affects grain yield under drought conditions.

### Example 12. BCAA Level is Significantly Accumulated in Gmbcat1/2 Plant

Previous studies have reported that Arabidopsis thaliana and rice increase BCAAs in response to drought and osmotic stress. Therefore, it was evaluated whether there were differences in BCAA levels in NT and Gmbcat1/2 mutant plants.

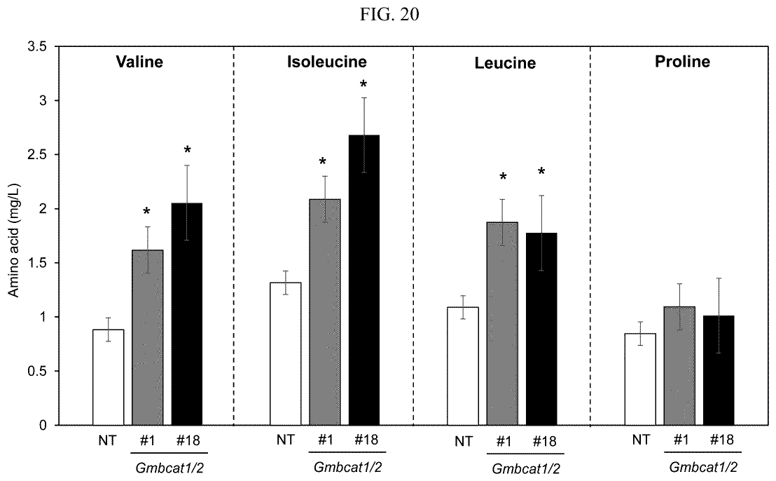

For HPLC analysis, leaves of 6-week-old plants were sampled under normal conditions, and the BCAA levels of NT plants and Gmbcat1/2 mutants were compared to each other. The results showed that BCAAs were accumulated more highly in Gmbcat1/2 than in NT. Specifically, Gmbcat1/2 mutants showed higher BCAA (Val, Ile, Leu) contents than NT plants (FIG. 20).

What is claimed is:

1. A method of enhancing tolerance to drought stress in a plant, the method comprising:
inhibiting expression of a gene encoding a protein consisting of the amino acid sequence of SEQ ID NO: 2 or genes encoding BCAT1 protein and BCAT2 protein from soybean consisting of the amino acid sequence of SEQ ID NO: 32 and SEQ ID NO: 34, respectively in a plant cell.

2. The method of claim 1, wherein the inhibiting of the expression is carried out by at least one of:
transforming a plant cell with a recombinant vector containing the gene encoding the protein;
knocking out the gene encoding the protein;
inserting T-DNA into the gene encoding the protein; and/or
inducing a mutation of the gene encoding the protein by an endogenous transposon or irradiation of X-ray or γ-ray.

3. The method of claim 1, wherein enhanced tolerance to drought stress is obtained by knocking-out the gene encoding the protein in the plant cell by using a gene editing system.

4. A method of producing a transgenic plant having enhanced tolerance to drought stress, the method comprising:
transforming a plant cell with a recombinant vector containing a gene encoding a protein consisting of the amino acid sequence of SEQ ID NO: 2 or genes encoding BCAT1 protein and BCAT2 protein from soybean consisting of the amino acid sequence of SEQ ID NO: 32 and SEQ ID NO: 34, respectively to inhibit expression of the gene encoding the protein in the plant cell; and
regenerating a transgenic plant from the transformed plant cell.

5. A transgenic plant having enhanced tolerance to drought stress produced by the method of claim 4.

6. A transgenic seed of the transgenic plant according to claim 5, wherein the transgenic seed comprises the recombinant vector.

7. A method of producing a genome-edited rice plant having enhanced tolerance to drought stress, the method comprising:
(a) carrying out genome editing by introducing guide RNA specific to target nucleotide sequence in a gene encoding a protein consisting of the amino acid sequence of SEQ ID NO: 2, and an endonuclease protein to a rice plant cell;
(b) producing a rice plant cell comprising an edited nucleotide sequence; and
(c) regenerating a plant from the genome-edited rice plant cell,
wherein the target nucleotide sequence is the nucleotide sequence of SEQ ID NO: 3 or SEQ ID NO: 4.

8. The method of claim 7, wherein the introduction of the guide RNA and the endonuclease protein to the rice plant cell further comprises:
using a recombinant vector containing a DNA which encodes guide RNA specific to target nucleotide sequence in the gene encoding the protein and a nucleic acid sequence which encodes the endonuclease protein: or a ribonucleoprotein between the guide RNA and the endonuclease protein.

9. A genome-edited rice plant having enhanced tolerance to drought stress produced by the method of claim 7.

10. A genome-edited seed of the rice plant according to claim 9, wherein the genome-edited seed comprises the edited target nucleotide sequence.

11. A method of producing a genome-edited soybean plant having enhanced tolerance to drought stress, the method comprising:
(a) carrying out genome editing by introducing guide RNA specific to target nucleotide sequence in genes encoding BCAT1 protein and BCAT2 protein from soybean consisting of the amino acid sequence of SEQ ID NO: 32 and SEQ ID NO: 34, respectively, and an endonuclease protein to a soybean plant cell;
(b) producing a soybean plant cell comprising an edited nucleotide sequence; and
(c) regenerating a plant from the genome-edited soybean plant cell,
wherein the target nucleotide sequence is the nucleotide sequence of SEQ ID NO: 35.

12. The method of claim 11, wherein the introduction of the guide RNA and the endonuclease protein to the soybean plant cell further comprises:
using a recombinant vector containing a DNA which encodes guide RNA specific to target nucleotide sequence in the genes encoding the proteins and a nucleic acid sequence which encodes the endonuclease protein: or a ribonucleoprotein between the guide RNA and the endonuclease protein.

13. A genome-edited soybean plant having enhanced tolerance to drought stress produced by the method of claim 11.

14. A genome-edited seed of the soybean plant according to claim 13, wherein the genome-edited seed comprises the edited target nucleotide sequence.
